(19) United States
(12) Patent Application Publication
(10) Pub. No.: US 2026/0306485 A1
(43) Pub. Date: Oct. 8, 2026

(54) COMPOSITIONS AND METHODS FOR THE TREATMENT OF CONGENITAL ADRENAL HYPERPLASIA

(71) Applicant: CRINETICS PHARMACEUTICALS, INC., San Diego, CA (US)

(72) Inventors: Alejandro AYALA, Sunny Isles Beach, FL (US); Alan KRASNER, Encinitas, CA (US); Sha LUO, San Diego, CA (US); Peter TRAINER, Wilmslow (GB)

(21) Appl. No.: 19/485,160

(22) PCT Filed: May 16, 2024
(86) PCT No.: PCT/US2024/029588

§ 371 Date: Nov. 17, 2025

Related U.S. Application Data
(60) Provisional application No. 63/503,248, filed on May 19, 2023.
(60) Provisional application No. 63/631,658, filed on Apr. 9, 2024.

(51) Int. Cl. A61K 31/496; A61K 31/573; A61P 5/08; A61P 5/28; A61P 15/00; A61P 17/10

(52) U.S. Cl. CPC A61K 31/496; A61K 31/573; A61P 5/08; A61P 5/28; A61P 15/00; A61P 17/10

(57) ABSTRACT

Provided are compositions and methods for the treatment of congenital adrenal hyperplasia (CAH).

### CROSS-REFERENCE TO RELATED APPLICATIONS

This application claims the benefit of U.S. Provisional Patent Application No. 63/503,248, filed on May 19, 2023; and U.S. Provisional Patent Application No. U.S. 63/631,658, filed on Apr. 9, 2024; each of which is incorporated herein by reference in its entirety.

### FIELD

Provided herein are compositions and methods for the treatment of congenital adrenal hyperplasia (CAH).

### BACKGROUND

Congenital adrenal hyperplasia (CAH) is a series of rare autosomal recessive diseases of the adrenal glands due to mutations in key enzymes involved in adrenal steroidogenesis.

The only currently available treatment for CAH in the United States is glucocorticoid therapy. However, the doses of glucocorticoids required to sufficiently suppress androgen production to effectively treat classic CAH are often higher than required for physiologic replacement. These high dosages result in the well-documented side-effect of chronic supraphysiologic glucocorticoid exposure, which is essentially an iatrogenic state of Cushing's syndrome (CS). Thus, there is a delicate balance between under- and overtreatment as patients with CAH often suffer chronically from various degrees of hyperandrogenism caused by the disease along with the manifold complications of chronic supraphysiologic glucocorticoid therapy used to treat the disease.

Under-replacement of glucocorticoids in CAH patients can result in low blood pressure, hyponatremia, and hyperkalemia due to absent mineralocorticoid; hypoglycemia due to decreased gluconeogenesis; reduced final adult height due to early epiphyseal closure due to increased adrenal androgens and increased bone mass; weight loss; and muscle weakness and myalgia. Over-replacement of glucocorticoids in CAH patients can result in high blood pressure, volume retention, edema, hypernatremia, and hypokalemia; hyperglycemia, hypertriglyceridemia due to increased gluconeogenesis and lipolysis; osteopenia, osteoporosis due to glucocorticoid-induced inhibition of osteoclast function, and vitamin D antagonism; increased fat mass (i.e., adrenocortical obesity) and predominantly of centripetal distribution; and muscle atrophy and myopathy.

As described above, the consequences of under- or over-replacement of glucocorticoids have serious implications on a multitude of bodily systems, including cardiovascular, liver, bone, adipose tissue, and muscle systems. As such, there remains an unmet need for new compositions and methods useful for the treatment of CAH that circumvent the drawbacks associated with glucocorticoid therapy. The present disclosure fulfills these and other needs, evident in reference to the following disclosure.

### BRIEF SUMMARY

Provided is a method of treating congenital adrenal hyperplasia (CAH) in a subject, said method comprising administering to the subject a compound of Formula (I), or a pharmaceutically acceptable salt thereof:

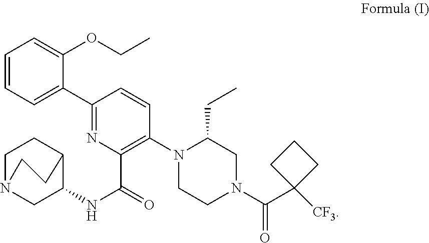 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is a method of treating hyperandrogenism in a subject with CAH, said method comprising administering to the subject a compound of Formula (I), or a pharmaceutically acceptable salt thereof:

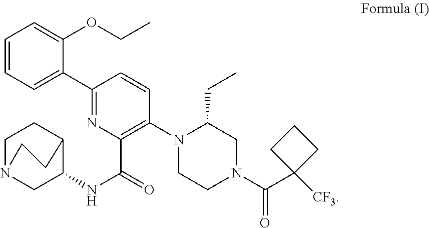 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

In certain embodiments, the compound of Formula (I), or the pharmaceutically acceptable salt thereof, is administered in a therapeutically effective amount.

In certain embodiments, the compound of Formula (I), or the pharmaceutically acceptable salt thereof, is administered in an amount equivalent to about 40 mg to about 160 mg of Formula (I), about 80 mg to about 160 mg of Formula (I), about 80 mg to about 120 mg of Formula (I), about 80 mg of Formula (I), about 120 mg of Formula (I), or about 160 mg of Formula (I).

In certain embodiments, the compound of Formula (I), or the pharmaceutically acceptable salt thereof, is administered in one or more unit dosage forms. In this embodiment, the unit dosage form can comprise the compound of Formula (I), or the pharmaceutically acceptable salt thereof, in an amount of equivalent to about 40 mg of Formula (I).

In certain embodiments, the unit dosage form is an oral unit dosage form.

In certain embodiments, the compound of Formula (I), or the pharmaceutically acceptable salt thereof, is administered daily. In this embodiment, the compound of Formula (I), or the pharmaceutically acceptable salt thereof, can be administered once daily.

In certain embodiments, the CAH is caused by 21-hydroxylase deficiency.

In certain embodiments, the subject is concurrently on glucocorticoid replacement therapy. In this embodiment, the subject can concurrently be on hydrocortisone, prednisolone, prednisone, or methylprednisolone therapy.

In certain embodiments, the subject is concurrently on mineralocorticoid replacement therapy. In this embodiment, the subject can concurrently be on fludrocortisone therapy.

In certain embodiments, treating comprises reducing the subject's serum levels of androstenedione (A4) from baseline. In this embodiment, the subject's serum levels of A4 can be reduced by >75% from baseline, >80% from baseline, >85% from baseline, or >90% from baseline.

In certain embodiments, treating comprises reducing the subject's serum levels of 17-hydroxyprogesterone (17-OHP) from baseline. In this embodiment, the subject's serum levels of 17-OHP can be reduced by >75% from baseline, >80% from baseline, >85% from baseline, >90% from baseline, or >95% from baseline.

In certain embodiments, treating comprises reducing the subject's serum levels of one or more of 11-deoxycorticosterone (11-DOC), 11β-hydroxyandrostenedione (11-OHA4), 11-ketoandrostenedione (11-ketoA4), 11β-hydroxytestosterone (11-OHT), and 11-ketotestosterone (11-ketoT) from baseline. In this embodiment, the baseline can be measured in the morning.

In certain embodiments, treating comprises reducing adrenal gland size, reducing waist circumference, or reducing levels of hemoglobin A1C (HbA1C).

In certain embodiments, the subject has hyperandrogenism.

In certain embodiments, the subject has acne. In this embodiment, subject can report their acne has improved following administration of the compound of Formula (I), or a pharmaceutically acceptable salt thereof.

In certain embodiments, the subject has hirsutism. In this embodiment, subject can report their hirsutism has improved following administration of the compound of Formula (I), or a pharmaceutically acceptable salt thereof.

In certain embodiments, the subject has menstrual dysfunction. In this embodiment, the subject can report their menstrual dysfunction has improved following administration of the compound of Formula (I), or a pharmaceutically acceptable salt thereof.

In certain embodiments, the subject has testicular adrenal rests tumor (TART). In this embodiment, the subject's TART can improve following administration of the compound of Formula (I), or a pharmaceutically acceptable salt thereof.

In certain embodiments, the subject is female. In this embodiment, treating comprises reducing the subject's serum levels of testosterone from baseline. In this embodiment, the subject's serum levels of testosterone can be reduced by >50% from baseline, >60% from baseline, >70% from baseline, or >80% from baseline.

Also provided is a pharmaceutical composition for use in treating a patient with CAH, said composition including a compound of Formula (I), or a pharmaceutically acceptable salt thereof:

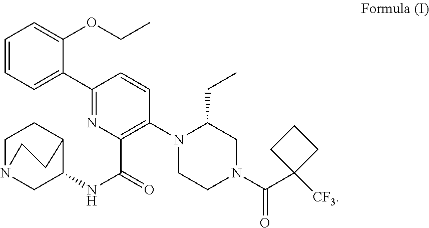 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is the use of a compound of Formula (I), or a pharmaceutically acceptable salt thereof, in the manufacture of a medicament for treating a patient with CAH:

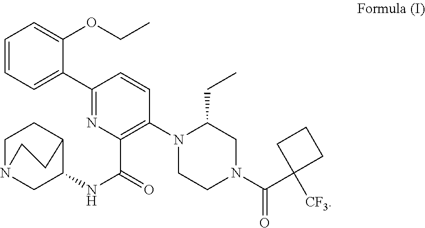 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is a pharmaceutical composition for use in treating hyperandrogenism in a patient with CAH, said composition including a compound of Formula (I), or a pharmaceutically acceptable salt thereof:

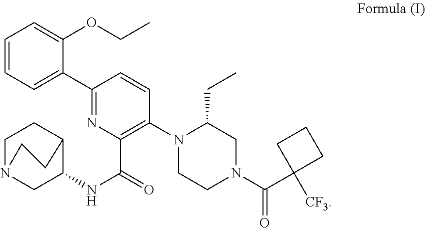 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is the use of a compound of Formula (I), or a pharmaceutically acceptable salt thereof, in the manufacture of a medicament for treating hyperandrogenism in a patient with CAH:

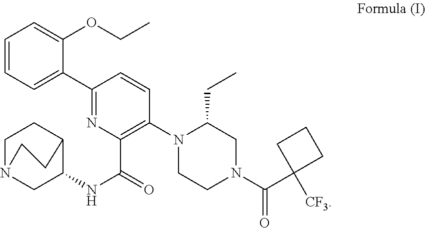 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

### BRIEF DESCRIPTION OF THE DRAWINGS

FIG. 1 depicts initial Cohort 1 analysis of the mean change from baseline standard error (S.E.) of morning serum androstenedione (A4) (ng/dL) wherein ULN represents the upper limit of normal.

FIG. 2 depicts initial Cohort 1 analysis of the mean change from baseline ±S.E. of morning serum 17-hydroxyprogesterone (17-OHP) (ng/dL).

FIG. 3 depicts initial Cohort 1 analysis of the mean change from baseline ±S.E. of morning plasma adrenocorticotropic hormone (ACTH) (pg/mL) wherein ULN represents the upper limit of normal.

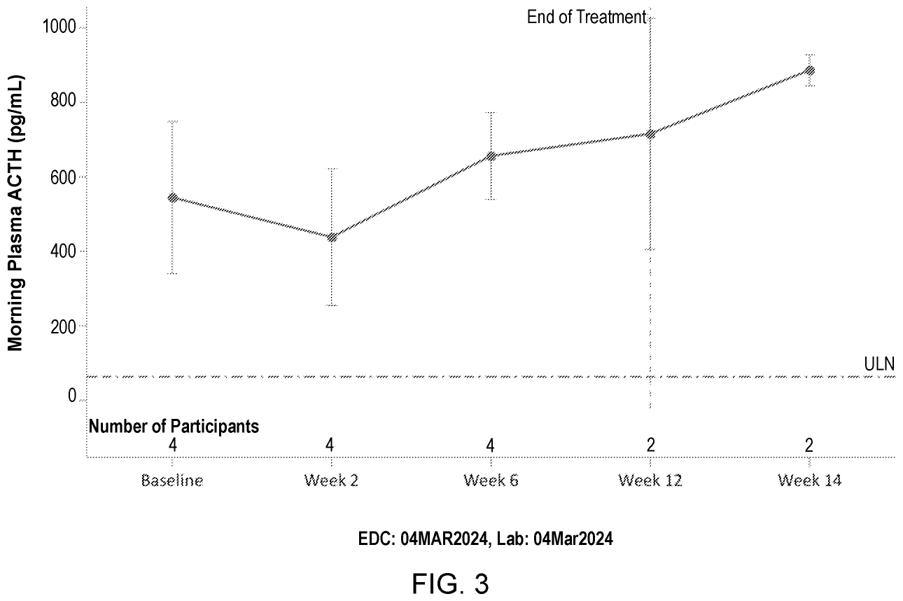

FIG. 4 depicts initial Cohort 1 analysis of the mean change from baseline ±S.E. of morning serum testosterone (ng/dL) of female (women) participants.

FIG. 5 depicts initial Cohort 1 analysis of the mean change from baseline ±S.E. of morning serum testosterone (ng/dL) of male (men) participants.

FIG. 6 depicts initial Cohort 1 analysis of the mean change from baseline ±S.E. of morning serum dehydroepiandrosterone (DHEA) (ng/mL).

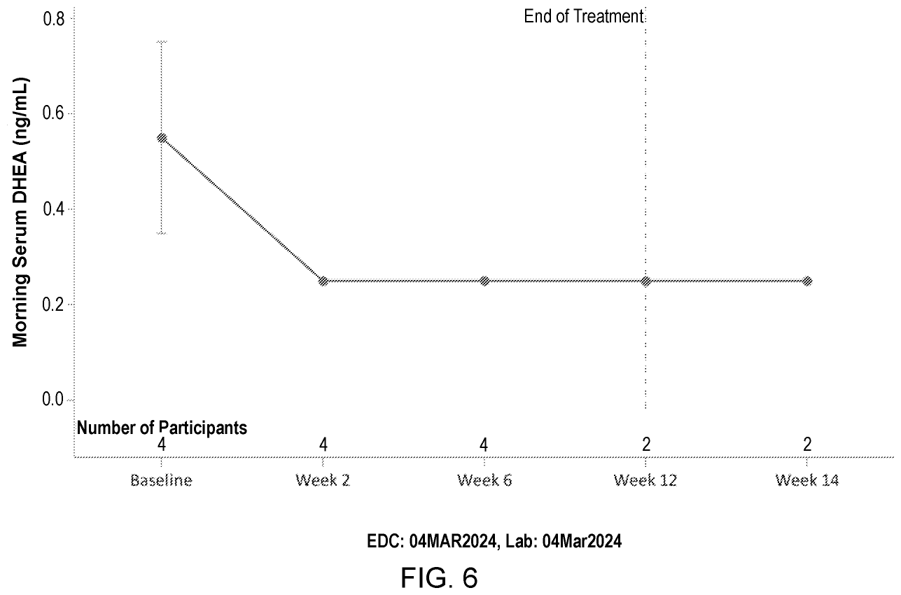

FIG. 7 depicts initial Cohort 1 analysis of the mean change from baseline ±S.E. of morning serum dehydroepiandrosterone sulfate (DHEA-S) (mcg/dL).

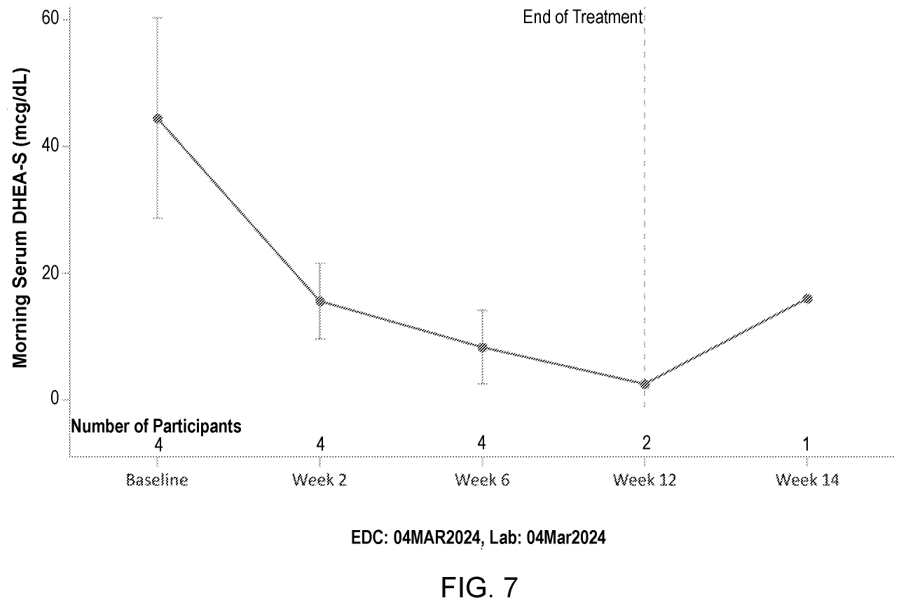

FIG. 8 depicts initial Cohort 1 analysis of the mean change from baseline S.E. of morning serum 11-deoxycorticosterone (ng/dL) wherein ULN represents the upper limit of normal.

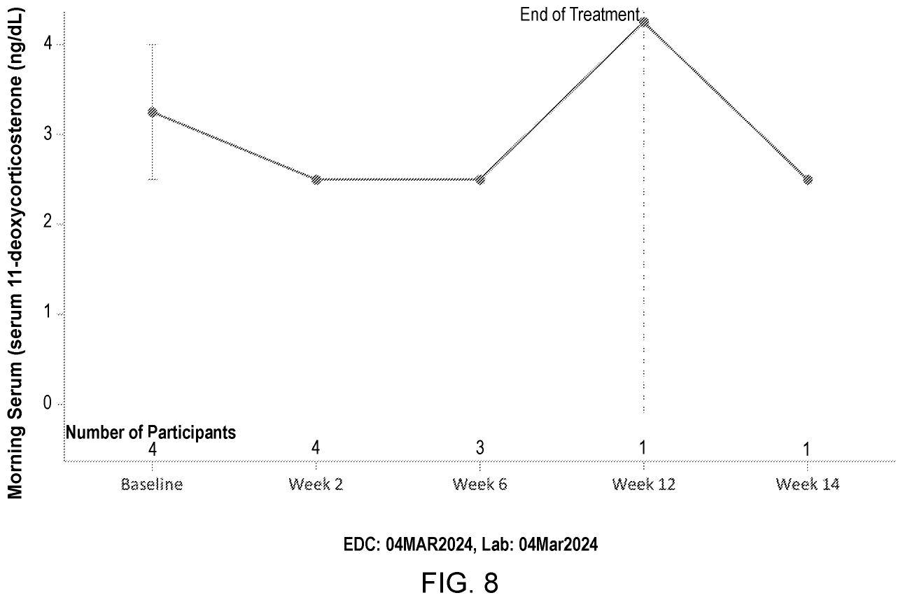

FIG. 9 depicts initial Cohort 1 analysis of the mean change from baseline S.E. of morning serum 21-deoxycortisol (ng/dL).

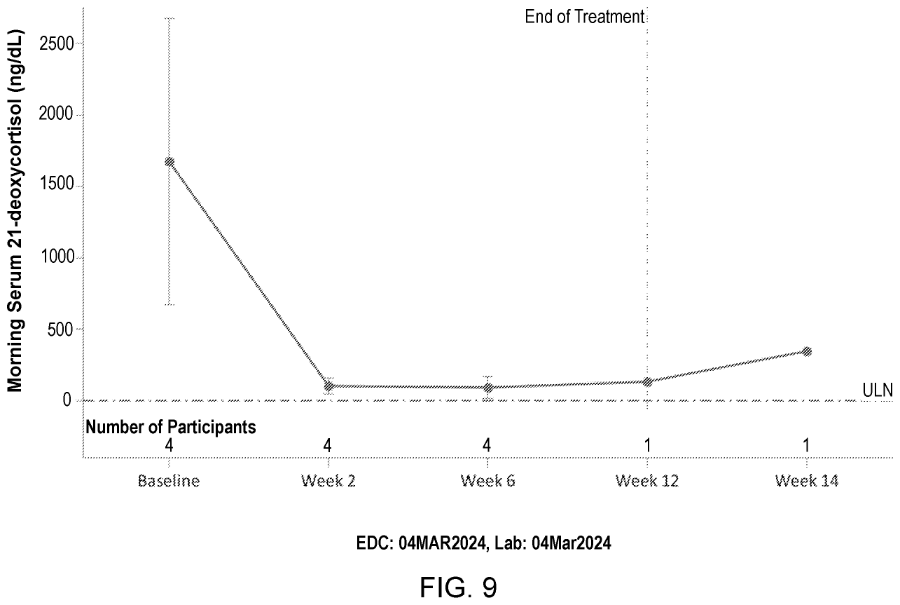

FIG. 10 depicts initial Cohort 1 analysis of the mean change from baseline S.E. of morning serum androstenedione (A4)/testosterone of male (men) participants.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as “comprises” and “comprising” are to be construed in an open, inclusive sense, that is, as “including, but not limited to.” Further, headings provided herein are for convenience only and do not interpret the scope or meaning of the claimed invention.

References throughout this specification to “one embodiment” or “an embodiment” or “some embodiments” or “a certain embodiment” means that a particular feature, structure, or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, “about” means±10% of the stated value, and includes more specifically values of ±5%, ±2%, and ±1% of the stated value.

As used herein, “administering to a patient” refers to the process of introducing a composition or dosage form into the patient via an art-recognized means of introduction.

As used herein the term “disorder” is intended to be generally synonymous, and is used interchangeably with the terms “disease,” “syndrome,” and “condition” (as in medical condition), in that all reflect an abnormal condition of the human or animal body or of one of its parts that impairs normal functioning, and is typically manifested by distinguishing signs and symptoms.

As used herein, Formula (I) refers to a compound having the structure shown below:

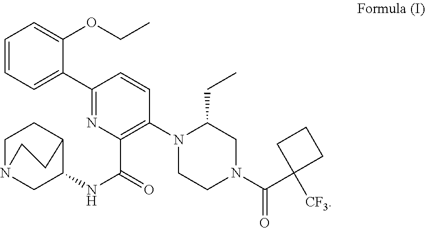 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

The compound of Formula (I) is also known as CRN04894, CRN04894 free base, N-[(3S)-1-Azabicyclo[2.2.2]octan-3-yl]-6-(2-ethoxyphenyl)-3-[(2R)-2-ethyl-4-[1-(trifluoromethyl)cyclobutanecarbonyl]-piperazin-1-yl]pyridine-2-carboxamide (IUPAC name), N-[(3S)-1-Azabicyclo[2.2.2]octan-3-yl]-6-(2-ethoxyphenyl)-3-[(2R)-2-ethyl-4-[1-(trifluoromethyl)cyclobutanecarbonyl]-piperazin-1-yl]pyridine-2-carboxamide free base, 6-(2-Ethoxyphenyl)-3-((R)-2-ethyl-4-(1-(trifluoromethyl)cyclobutane-1-carbonyl)piperazin-1-yl)-N—((S)-quinuclidin-3-yl)picolinamide, and 6-(2-Ethoxyphenyl)-3-((R)-2-ethyl-4-(1-(trifluoromethyl)cyclobutane-1-carbonyl)piperazin-1-yl)-N—((S)-quinuclidin-3-yl)picolinamide free base.

As used herein, a “dose” means the measured quantity of an active agent to be taken at one time by a patient. In certain embodiments, wherein the active agent is not the free base of the compound of Formula (I), the quantity is the molar equivalent to the corresponding amount of the free base of the compound of Formula (I). For example, often a drug is packaged in a pharmaceutically acceptable salt form, such as the maleate salt of the compound of Formula (I), and the dosage form strength refers to the mass of the molar equivalent of the corresponding free base. As an example, 47.56 mg of the maleate salt of the compound of Formula (I) is the molar equivalent of 40.00 mg of the free base of the compound of Formula (I).

As used herein, “effective amount” and “therapeutically effective amount” of an agent, compound, drug, or composition is an amount which is nontoxic and effective for producing some desired therapeutic effect upon administration to a subject or patient (e.g., a human subject or patient).

As used herein, “patient” or “individual” or “subject” means a mammal, including a human, for whom or which therapy is desired, and generally refers to the recipient of the therapy.

As used herein, “pharmaceutically acceptable” refers to a material that is not biologically or otherwise undesirable, i.e., the material may be incorporated into a pharmaceutical composition to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with suitable inorganic acids, inorganic bases, or organic bases.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “treating” or “treatment” refers to therapeutic applications to slow or stop progression of a disorder, and/or alleviate symptoms thereof.

### Methods of Treatment

Provided is a method of treating congenital adrenal hyperplasia (CAH) in a subject comprising administering to the subject a compound of Formula (I), or a pharmaceutically acceptable salt thereof:

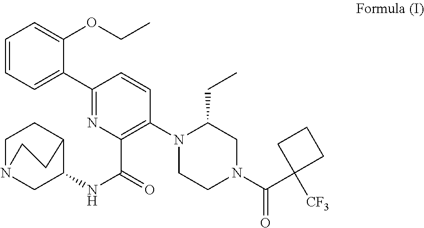 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is a method of treating hyperandrogenism in a subject with CAH comprising administering to the subject a compound of Formula (I), or a pharmaceutically acceptable salt thereof:

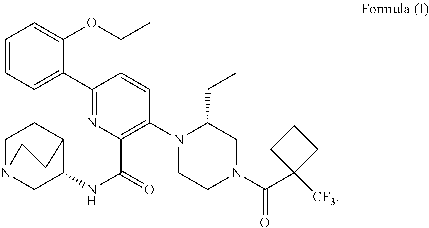 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

In certain embodiments, the compound of Formula (I), or a pharmaceutically acceptable salt thereof, is administered in a therapeutically effective amount.

In certain embodiments, the compound of Formula (I), or a pharmaceutically acceptable salt thereof, is administered in an amount equivalent to about 40 mg to about 160 mg of Formula (I). In certain embodiments, the compound of Formula (I), or a pharmaceutically acceptable salt thereof, is administered in an amount equivalent to about 80 mg to about 160 mg of Formula (I). In certain embodiments, the compound of Formula (I), or a pharmaceutically acceptable salt thereof, is administered in an amount equivalent to about 80 mg to about 120 mg of Formula (I).

In certain embodiments, the compound of Formula (I), or a pharmaceutically acceptable salt thereof, is administered in an amount equivalent to about 40 mg of Formula (I). In certain embodiments, the compound of Formula (I), or a pharmaceutically acceptable salt thereof, is administered in an amount equivalent to about 80 mg of Formula (I). In certain embodiments, the compound of Formula (I), or a pharmaceutically acceptable salt thereof, is administered in an amount equivalent to about 120 mg of Formula (I). In certain embodiments, the compound of Formula (I), or a pharmaceutically acceptable salt thereof, is administered in an amount equivalent to about 160 mg of Formula (I).

In certain embodiments, the compound of Formula (I), or a pharmaceutically acceptable salt thereof, is administered in one or more unit dosage forms. In certain embodiments, the compound of Formula (I), or a pharmaceutically acceptable salt thereof, is administered in two or more unit dosage forms. In certain embodiments, the compound of Formula (I), or a pharmaceutically acceptable salt thereof, is administered in three or more unit dosage forms. In certain embodiments, the compound of Formula (I), or a pharmaceutically acceptable salt thereof, is administered in four or more unit dosage forms.

In certain embodiments, the unit dosage form comprises an amount of the compound of Formula (I), or a pharmaceutically acceptable salt thereof, equivalent to about 40 mg of Formula (I).

In certain embodiments, the compound of Formula (I), or a pharmaceutically acceptable salt thereof, is administered in two unit dosage forms and each unit dosage form comprises an amount of the compound of Formula (I), or a pharmaceutically acceptable salt thereof, equivalent to about 40 mg of Formula (I). In certain embodiments, the compound of Formula (I), or a pharmaceutically acceptable salt thereof, is administered in three unit dosage forms and each unit dosage form comprises an amount of the compound of Formula (I), or a pharmaceutically acceptable salt thereof, equivalent to about 40 mg of Formula (I). In certain embodiments, the compound of Formula (I), or a pharmaceutically acceptable salt thereof, is administered in four unit dosage forms and each unit dosage form comprises an amount of the compound of Formula (I), or a pharmaceutically acceptable salt thereof, equivalent to about 40 mg of Formula (I).

In certain embodiments, the unit dosage form is an oral unit dosage form.

In certain embodiments, the compound of Formula (I), or a pharmaceutically acceptable salt thereof, is administered daily. In certain embodiments, the compound of Formula (I), or a pharmaceutically acceptable salt thereof, is administered once daily. In embodiments where the compound of Formula (I), or a pharmaceutically acceptable salt thereof, is administered in two or more unit dosage forms (i.e., two, three, or four unit dosage forms), once daily administration encompasses administering all unit dosage forms simultaneously, or administering the first unit dosage form and immediately administering each additional unit dosage form thereafter until all unit dosage forms have been administered to the subject.

In certain embodiments, the CAH is caused by 21-hydroxylase deficiency.

In certain embodiments, the subject is concurrently on glucocorticoid replacement therapy. In certain embodiments, the subject is concurrently on hydrocortisone, prednisolone, prednisone, or methylprednisolone therapy. In certain embodiments, the subject is concurrently on hydrocortisone therapy. In certain embodiments, the subject is concurrently on prednisolone therapy. In certain embodiments, the subject is concurrently on prednisone therapy. In certain embodiments, the subject is concurrently on methylprednisolone therapy.

In certain embodiments, the subject is concurrently on mineralocorticoid replacement therapy. In certain embodiments, the subject is concurrently on fludrocortisone therapy.

In certain embodiments, treating comprises reducing the subject's serum levels of androstenedione (A4) from baseline. In certain embodiments, the subject's serum levels of A4 are reduced by >25% from baseline. In certain embodiments, the subject's serum levels of A4 are reduced by >50% from baseline. In certain embodiments, the subject's serum levels of A4 are reduced by >75% from baseline. In certain embodiments, the subject's serum levels of A4 are reduced by >80% from baseline. In certain embodiments, the subject's serum levels of A4 are reduced by >85% from baseline. In certain embodiments, the subject's serum levels of A4 are reduced by >90% from baseline.

In certain embodiments, the reduction in the subject's serum levels of A4 from baseline is maintained for at least one week following discontinuation of treatment with CRN04894. In certain embodiments, the reduction in the subject's serum levels of A4 from baseline is maintained for at least two weeks following discontinuation of treatment with CRN04894. In certain embodiments, the reduction in the subject's serum levels of A4 from baseline is maintained for at least three weeks following discontinuation of treatment with CRN04894. In certain embodiments, the reduction in the subject's serum levels of A4 from baseline is maintained for at least four weeks following discontinuation of treatment with CRN04894. In certain embodiments, the reduction in the subject's serum levels of A4 from baseline is maintained throughout the course of treatment with CRN04894.

In certain embodiments, the subject has a baseline serum A4 level greater than or equal to 1.2 times the upper limit of normal. In certain embodiments, the subject has a baseline serum A4 level greater than or equal to 1.5 times the upper limit of normal. In certain embodiments, the upper limit of normal is about 150 ng/dL for adult males and about 200 ng/dL for adult females. In certain embodiments, the upper limit of normal is about 140 ng/dL for adolescent males of at least 16 years of age and about 210 ng/dL for adolescent males of at least 17 years of age. In certain embodiments, the upper limited of normal is about 240 ng/dL for adolescent females of at least 16 years of age.

In certain embodiments, treating comprises reducing the subject's serum levels of 17-hydroxyprogesterone (17-OHP) from baseline. In certain embodiments, the subject's serum levels of 17-OHP are reduced by >25% from baseline. In certain embodiments, the subject's serum levels of 17-OHP are reduced by >50% from baseline. In certain embodiments, the subject's serum levels of 17-OHP are reduced by >75% from baseline. In certain embodiments, the subject's serum levels of 17-OHP are reduced by >80% from baseline. In certain embodiments, the subject's serum levels of 17-OHP are reduced by >85% from baseline. In certain embodiments, the subject's serum levels of 17-OHP are reduced by >90% from baseline. In certain embodiments, the subject's serum levels of 17-OHP are reduced by >95% from baseline.

In certain embodiments, the reduction in the subject's serum levels of 17-OHP from baseline is maintained for at least one week following discontinuation of treatment with CRN04894. In certain embodiments, the reduction in the subject's serum levels of 17-OHP from baseline is maintained for at least two weeks following discontinuation of treatment with CRN04894. In certain embodiments, the reduction in the subject's serum levels of 17-OHP from baseline is maintained for at least three weeks following discontinuation of treatment with CRN04894. In certain embodiments, the reduction in the subject's serum levels of 17-OHP from baseline is maintained for at least four weeks following discontinuation of treatment with CRN04894. In certain embodiments, the reduction in the subject's serum levels of 17-OHP from baseline is maintained throughout the course of treatment with CRN04894.

In certain embodiments, the subject has a baseline 17-OHP serum level of greater than or equal to 1,000 ng/dL. In certain embodiments, the subject has a baseline 17-OHP serum level of greater than or equal to 30.3 nmol/L.

In certain embodiments, treating comprises reducing the subject's serum levels of one or more of 11-deoxycorticosterone (11-DOC), 11β-hydroxyandrostenedione (11-OHA4), 11-ketoandrostenedione (11-ketoA4), 11β-hydroxytestosterone (11-OHT), and 11-ketotestosterone (11-ketoT) from baseline.

In certain embodiments, the baseline is measured in the morning.

In certain embodiments, treating comprises reducing adrenal gland size.

In certain embodiments, treating comprises reducing waist circumference.

In certain embodiments, treating comprises reducing levels of hemoglobin A1C (HbA1C).

In certain embodiments, the subject has hyperandrogenism.

In certain embodiments, the subject has acne. In certain embodiments, the subject reports their acne has improved following administration of the compound of Formula (I), or a pharmaceutically acceptable salt thereof. For example, the subject can report their acne has improved on a patient questionnaire.

In certain embodiments, the subject has hirsutism. In certain embodiments, the subject reports their hirsutism has improved following administration of the compound of Formula (I), or a pharmaceutically acceptable salt thereof. For example, the subject can report their hirsutism has improved on a patient questionnaire.

In certain embodiments, the subject has menstrual dysfunction. In certain embodiments, the subject reports their menstrual dysfunction has improved following administration of the compound of Formula (I), or a pharmaceutically acceptable salt thereof. For example, the subject can report their menstrual dysfunction has improved on a patient questionnaire or in a menstrual cycle diary.

In certain embodiments, the subject has testicular adrenal rests tumor (TART). In certain embodiments, the subject's TART has improved following administration of the compound of Formula (I), or a pharmaceutically acceptable salt thereof. In certain embodiments, improvements in the subject's TART include, but are not limited to, reducing testicular pain, reducing the number of tumors, reducing the size of one or more tumors, or preventing the occurrence of additional tumors.

In certain embodiments, the subject is a human. In certain embodiments, the human is an adult. In certain embodiments, the human is an adolescent.

In certain embodiments, treating comprises reducing the subject's serum levels of testosterone from baseline. In certain embodiments, the subject's serum levels of testosterone are reduced by >50% from baseline. In certain embodiments, the subject's serum levels of testosterone are reduced by >60% from baseline. In certain embodiments, the subject's serum levels of testosterone are reduced by >70% from baseline. In certain embodiments, the subject's serum levels of testosterone are reduced by >80% from baseline.

In certain embodiments, the subject is female. In certain embodiments, treating comprises reducing the female subject's serum levels of testosterone from baseline. In certain embodiments, the female subject's serum levels of testosterone are reduced by >50% from baseline. In certain embodiments, the female subject's serum levels of testosterone are reduced by >60% from baseline. In certain embodiments, the female subject's serum levels of testosterone are reduced by >70% from baseline. In certain embodiments, the female subject's serum levels of testosterone are reduced by >80% from baseline.

In certain embodiments, the reduction in the subject's serum levels of testosterone from baseline is maintained for at least one week following discontinuation of treatment with CRN04894. In certain embodiments, the reduction in the subject's serum levels of testosterone from baseline is maintained for at least two weeks following discontinuation of treatment with CRN04894. In certain embodiments, the reduction in the subject's serum levels of testosterone from baseline is maintained for at least three weeks following discontinuation of treatment with CRN04894. In certain embodiments, the reduction in the subject's serum levels of testosterone from baseline is maintained for at least four weeks following discontinuation of treatment with CRN04894. In certain embodiments, the reduction in the subject's serum levels of testosterone from baseline is maintained throughout the course of treatment with CRN04894.

### Pharmaceutical Compositions

Provided is a pharmaceutical composition for use in treating a subject with congenital adrenal hyperplasia (CAH), wherein the composition comprises a compound of Formula (I), or a pharmaceutically acceptable salt thereof:

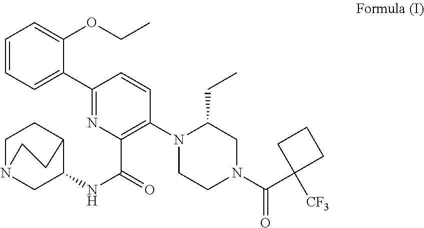 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

Also provided is a pharmaceutical composition for use in treating hyperandrogenism in a subject with CAH, wherein the composition comprises a compound of Formula (I), or a pharmaceutically acceptable salt thereof:

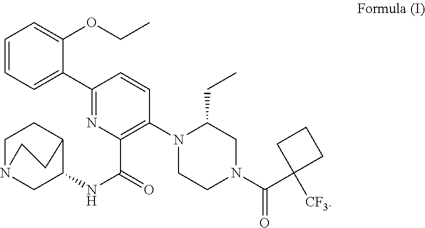 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

In certain embodiments, the pharmaceutical composition comprises a therapeutically effective amount of compound of Formula (I), or a pharmaceutically acceptable salt thereof.

In certain embodiments, the pharmaceutical composition contains an amount of the compound of Formula (I), or a pharmaceutically acceptable salt thereof, equivalent to about 40 mg to about 160 mg of Formula (I). In certain embodiments, the pharmaceutical composition contains an amount of the compound of Formula (I), or a pharmaceutically acceptable salt thereof, equivalent to about 80 mg to about 160 mg of Formula (I). In certain embodiments, the pharmaceutical composition contains an amount of the compound of Formula (I), or a pharmaceutically acceptable salt thereof, equivalent to about 80 mg to about 120 mg of Formula (I).

In certain embodiments, the pharmaceutical composition contains an amount of the compound of Formula (I), or a pharmaceutically acceptable salt thereof, equivalent to about 40 mg of Formula (I). In certain embodiments, the pharmaceutical composition contains an amount of the compound of Formula (I), or a pharmaceutically acceptable salt thereof, equivalent to about 80 mg of Formula (I). In certain embodiments, the pharmaceutical composition contains an amount of the compound of Formula (I), or a pharmaceutically acceptable salt thereof, equivalent to about 120 mg of Formula (I). In certain embodiments, the pharmaceutical composition contains an amount of the compound of Formula (I), or a pharmaceutically acceptable salt thereof, equivalent to about 160 mg of Formula (I).

In certain embodiments, the pharmaceutical compositions provided herein may be provided in unit dosage or multiple-dosage forms. Unit-dosage forms, as used herein, refer to physically discrete units suitable for administration to subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include individually packaged tablets. Unit dosage forms may be administered in fractions or multiples thereof. A multiple-dosage form is a plurality of identical unit-dosage forms packaged in a single container to be administered in segregated unit-dosage form. Examples of multiple-dosage forms include bottles of tablets.

In certain embodiments, the pharmaceutical composition is a unit dosage form that contains an amount of the compound of Formula (I), or a pharmaceutically acceptable salt thereof, equivalent to about 40 mg of Formula (I).

In certain embodiments, the pharmaceutical composition is a unit dosage form that is an oral unit dosage form.

In certain embodiments, the pharmaceutical composition is administered daily. In certain embodiments, the pharmaceutical composition is administered once daily.

In certain embodiments, the pharmaceutical composition further comprises one or more pharmaceutically acceptable carriers or excipients. Pharmaceutically acceptable carriers or excipients include, but are not limited to, binders, fillers, diluents, disintegrants, wetting agents, lubricants, glidants, coloring agents, dye-migration inhibitors, sweetening agents, and flavoring agents.

In certain embodiments, the CAH is caused by 21-hydroxylase deficiency.

In certain embodiments, the subject is concurrently on glucocorticoid replacement therapy. In certain embodiments, the subject is concurrently on hydrocortisone, prednisolone, prednisone, or methylprednisolone therapy. In certain embodiments, the subject is concurrently on hydrocortisone therapy. In certain embodiments, the subject is concurrently on prednisolone therapy. In certain embodiments, the subject is concurrently on prednisone therapy. In certain embodiments, the subject is concurrently on methylprednisolone therapy.

In certain embodiments, the subject is concurrently on mineralocorticoid replacement therapy. In certain embodiments, the subject is concurrently on fludrocortisone therapy.

In certain embodiments, treating comprises reducing the subject's serum levels of androstenedione (A4) from baseline. In certain embodiments, the subject's serum levels of A4 are reduced by >25% from baseline. In certain embodiments, the subject's serum levels of A4 are reduced by >50% from baseline. In certain embodiments, the subject's serum levels of A4 are reduced by >75% from baseline. In certain embodiments, the subject's serum levels of A4 are reduced by >80% from baseline. In certain embodiments, the subject's serum levels of A4 are reduced by >85% from baseline. In certain embodiments, the subject's serum levels of A4 are reduced by >90% from baseline.

In certain embodiments, the reduction in the subject's serum levels of A4 from baseline is maintained for at least one week following discontinuation of treatment with CRN04894. In certain embodiments, the reduction in the subject's serum levels of A4 from baseline is maintained for at least two weeks following discontinuation of treatment with CRN04894. In certain embodiments, the reduction in the subject's serum levels of A4 from baseline is maintained for at least three weeks following discontinuation of treatment with CRN04894. In certain embodiments, the reduction in the subject's serum levels of A4 from baseline is maintained for at least four weeks following discontinuation of treatment with CRN04894. In certain embodiments, the reduction in the subject's serum levels of A4 from baseline is maintained throughout the course of treatment with CRN04894.

In certain embodiments, the subject has a baseline serum A4 level greater than or equal to 1.2 times the upper limit of normal. In certain embodiments, the subject has a baseline serum A4 level greater than or equal to 1.5 times the upper limit of normal. In certain embodiments, the upper limit of normal is about 150 ng/dL for adult males and about 200 ng/dL for adult females. In certain embodiments, the upper limit of normal is about 140 ng/dL for adolescent males of at least 16 years of age and about 210 ng/dL for adolescent males of at least 17 years of age. In certain embodiments, the upper limited of normal is about 240 ng/dL for adolescent females of at least 16 years of age.

In certain embodiments, treating comprises reducing the subject's serum levels of 17-hydroxyprogesterone (17-OHIP) from baseline. In certain embodiments, the subject's serum levels of 17-OHP are reduced by >25% from baseline. In certain embodiments, the subject's serum levels of 17-OHP are reduced by >50% from baseline. In certain embodiments, the subject's serum levels of 17-OHP are reduced by >75% from baseline. In certain embodiments, the subject's serum levels of 17-OHP are reduced by >80% from baseline. In certain embodiments, the subject's serum levels of 17-OHP are reduced by >85% from baseline. In certain embodiments, the subject's serum levels of 17-OHP are reduced by >90% from baseline. In certain embodiments, the subject's serum levels of 17-OHP are reduced by >95% from baseline.

In certain embodiments, the reduction in the subject's serum levels of 17-OHP from baseline is maintained for at least one week following discontinuation of treatment with CRN04894. In certain embodiments, the reduction in the subject's serum levels of 17-OHP from baseline is maintained for at least two weeks following discontinuation of treatment with CRN04894. In certain embodiments, the reduction in the subject's serum levels of 17-OHP from baseline is maintained for at least three weeks following discontinuation of treatment with CRN04894. In certain embodiments, the reduction in the subject's serum levels of 17-OHP from baseline is maintained for at least four weeks following discontinuation of treatment with CRN04894. In certain embodiments, the reduction in the subject's serum levels of 17-OHP from baseline is maintained throughout the course of treatment with CRN04894.

In certain embodiments, the subject has a baseline 17-OHP serum level of greater than or equal to 1,000 ng/dL. In certain embodiments, the subject has a baseline 17-OHP serum level of greater than or equal to 30.3 nmol/L.

In certain embodiments, treating comprises reducing the subject's serum levels of one or more of 11-deoxycorticosterone (11-DOC), 11β-hydroxyandrostenedione (11-OHA4), 11-ketoandrostenedione (11-ketoA4), 11β-hydroxytestosterone (11-OHT), and 11-ketotestosterone (11-ketoT) from baseline.

In certain embodiments, the baseline is measured in the morning.

In certain embodiments, treating comprises reducing adrenal gland size.

In certain embodiments, treating comprises reducing waist circumference.

In certain embodiments, treating comprises reducing levels of hemoglobin A1C (HbA1C).

In certain embodiments, the subject has hyperandrogenism.

In certain embodiments, the subject has acne. In certain embodiments, the subject reports their acne has improved following administration of the compound of Formula (I), or a pharmaceutically acceptable salt thereof. For example, the subject can report their acne has improved on a patient questionnaire.

In certain embodiments, the subject has hirsutism. In certain embodiments, the subject reports their hirsutism has improved following administration of the compound of Formula (I), or a pharmaceutically acceptable salt thereof. For example, the subject can report their hirsutism has improved on a patient questionnaire.

In certain embodiments, the subject has menstrual dysfunction. In certain embodiments, the subject reports their menstrual dysfunction has improved following administration of the compound of Formula (I), or a pharmaceutically acceptable salt thereof. For example, the subject can report their menstrual dysfunction has improved on a patient questionnaire or in a menstrual cycle diary.

In certain embodiments, the subject has testicular adrenal rests tumor (TART). In certain embodiments, the subject's TART has improved following administration of the compound of Formula (I), or a pharmaceutically acceptable salt thereof. In certain embodiments, improvements in the subject's TART include, but are not limited to, reducing testicular pain, reducing the number of tumors, reducing the size of one or more tumors, or preventing the occurrence of additional tumors.

In certain embodiments, the subject is a human. In certain embodiments, the human is an adult. In certain embodiments, the human is an adolescent.

In certain embodiments, treating comprises reducing the subject's serum levels of testosterone from baseline. In certain embodiments, the subject's serum levels of testosterone are reduced by >50% from baseline. In certain embodiments, the subject's serum levels of testosterone are reduced by >60% from baseline. In certain embodiments, the subject's serum levels of testosterone are reduced by >70% from baseline. In certain embodiments, the subject's serum levels of testosterone are reduced by >80% from baseline.

In certain embodiments, the subject is female. In certain embodiments, treating comprises reducing the female subject's serum levels of testosterone from baseline. In certain embodiments, the female subject's serum levels of testosterone are reduced by >50% from baseline. In certain embodiments, the female subject's serum levels of testosterone are reduced by >60% from baseline. In certain embodiments, the female subject's serum levels of testosterone are reduced by >70% from baseline. In certain embodiments, the female subject's serum levels of testosterone are reduced by >80% from baseline.

In certain embodiments, the reduction in the subject's serum levels of testosterone from baseline is maintained for at least one week following discontinuation of treatment with CRN04894. In certain embodiments, the reduction in the subject's serum levels of testosterone from baseline is maintained for at least two weeks following discontinuation of treatment with CRN04894. In certain embodiments, the reduction in the subject's serum levels of testosterone from baseline is maintained for at least three weeks following discontinuation of treatment with CRN04894. In certain embodiments, the reduction in the subject's serum levels of testosterone from baseline is maintained for at least four weeks following discontinuation of treatment with CRN04894. In certain embodiments, the reduction in the subject's serum levels of testosterone from baseline is maintained throughout the course of treatment with CRN04894.

### Congenital Adrenal Hyperplasia (CAH)

Congenital adrenal hyperplasia (CAH) is a series of rare autosomal recessive diseases of the adrenal glands due to mutations in key enzymes involved in adrenal steroidogenesis. The most common cause of CAH is 21-hydroxylase deficiency (21-OHD), accounting for over 90% of cases of CAH. This “classic form” of CAH is due to complete loss of 21-OH activity and has a worldwide incidence ranging from ~1:14,000 to 1:18,000.

The diagnosis of classic 21-OH deficiency is based on elevated levels of the immediate enzyme substrate 17-hydroxyprogesterone (17-OHP). Screening is routinely performed in newborn screening programs by measuring 17-OHIP. An elevated level is sensitive for CAH, but falsely high 17-OHP levels can occur in premature, sick, and stressed infants. Second-tier screening can confirm elevated levels seen on the newborn screen. In cases where the data are equivocal, a stimulation test demonstrating an exaggerated rise in 17-OHP in response to an exogenous dose of ACTH may be required.

Classic CAH due to 2-OHD deficiency results in a lack of cortisol and aldosterone production, which can cause salt-wasting adrenal crises in the neonate and life-long adrenal insufficiency. In the absence of cortisol, the hypothalamus and pituitary gland are not subject to negative feedback, resulting in elevated adrenocorticotropic hormone (ACTH) levels, which in turn drives excess adrenal androgen production. This excess ACTH acts on the adrenal glands resulting in bilateral adrenal hyperplasia and, due to 21-OH deficiency, elevated steroid precursors, such as androstenedione (A4), which can cause clinical hyperandrogenism.

In females, hyperandrogenism can result in hirsutism, acne, male-pattern baldness, menstrual irregularities, oligomenorrhea or amenorrhea, infertility, short stature, and frank virilization, manifesting in the newborn as ‘intersex.’

Poorly controlled childhood CAH results in short stature. Childhood hyperandrogenism in boys results in premature puberty/adrenarche with resultant premature fusion of the epiphyses. In men, hyperandrogenism can suppress gonadotropin secretion, resulting in reduced testes size, reduced testicular testosterone secretion, and spermatogenesis. Furthermore, chronic ACTH stimulation may also contribute to the formation of testicular adrenal rest tumors (TART) in men, which can be confused with malignant testicular tumors, and cause irreversible testicular damage and potential infertility. There are no therapies to prevent the development of TART other than intensified glucocorticoid treatment.

Current treatment of CAH centers on replacing deficient adrenal hormones (i.e., cortisol and aldosterone) and suppressing excess androgen production. Given that there are no other approved nonhormonal medications for CAH in the United States (US), glucocorticoid therapy must not only serve to replace the deficiency, but also provide feedback inhibition at the pituitary to decrease ACTH secretion and thereby reduce adrenal androgen production.

### CRN04894

CRN04894, i.e. the compound of Formula (I), is an oral, first-in-class adrenocorticotropic hormone (ACTH) receptor antagonist that binds to the melanocortin 2 receptor (MC2R) to block ACTH-mediated steroidogenesis from the adrenal gland. The structure of the compound of Formula (I) is shown below:

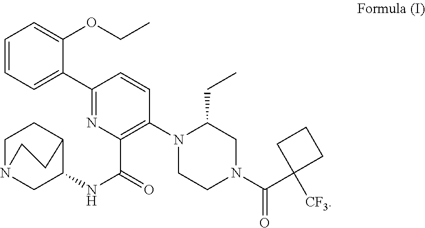 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

ACTH is a 39-amino acid peptide synthesized by anterior pituitary corticotropic cells and is secreted in response to hypothalamic corticotrophin releasing hormone (CRH) and stressful stimuli. As part of the hypothalamic-pituitary-adrenal (HPA) axis, ACTH activates the melanocortin 2 receptor (MC2R) on the adrenal gland. MC2R is expressed on the adrenal cortex and requires the MC2R accessory protein (MRAP) for both surface expression and binding to ACTH. Binding of ACTH to the MC2R/MRAP complex on adrenal cortical cells activates its G-protein (Gs) to elevate intracellular cyclic adenosine monophosphate (cAMP) levels, which in turn stimulate cortisol synthesis and secretion by regulating multiple steps in the steroidogenic pathway.

By acting selectively at MC2R, CRN04894 is a potential novel treatment for diseases that are characterized by ACTH-mediated pathological elevations of adrenal steroid hormones. This predicted ability to suppress adrenally derived androgens while maintaining mineralocorticoid production (due to lack of activity on angiotensin type 2 [AT II] receptor) makes CRN04894 a novel treatment option for CAH.

In conjunction, CRN04894 may also be an effective treatment for hyperandrogenism observed in CAH subjects, and may result in improvement in the associated problems, such as acne; hirsutism and menstrual dysfunction in women; and TART in men.

CRN04894, including the synthesis thereof, is disclosed in U.S. Pat. No. 11,566,015, the entire contents of which is incorporated by reference.

### EXAMPLES

### Example 1: A 12-Week, Phase 2 Open-Label, Sequential Dose Cohort Study to Evaluate the Safety, Efficacy, and Pharmacokinetics of CRN04894 Treatment in Participants with CAH

### Study Objectives and Endpoints

The primary efficacy objective is to evaluate the efficacy of CRN04894, measured by the change from baseline in serum androstenedione (A4), with the primary efficacy endpoint being the change from baseline in the morning (before 11:00 AM) serum A4 at week 12.

The secondary efficacy objective is to evaluate the efficacy of CRN04894, measured by the change from baseline in serum 17-hydroxyprogesterone (17-OHP), with the secondary efficacy endpoint being the change from baseline in the morning (before 11:00 AM) serum 17-OHP at week 12.

The safety objective is to evaluate the safety and tolerability of CRN04894, based on the incidence of treatment-emergent adverse events (TEAEs), including treatment-emergent serious adverse events (SAEs) and any adverse events (AEs) leading to discontinuation.

### Inclusion Criteria

Participants with congenital adrenal hyperplasia (CAH) caused by 21-hydroxylase deficiency will be recruited for this study.

Participants must be willing and able to understand and adhere to the study procedures.

Participants will include both male and female participants of 18 to 75 years of age. Participants of 16 years of age may be included in sites located in the United States. Classic 21-hydroxylase deficiency will be confirmed by the Investigator and approved by the Medical Monitor.

Participants will be on a stable (defined as no dose change of >5 mg/day hydrocortisone equivalent within 6 months prior to Screening) regimen of glucocorticoid replacement (e.g., hydrocortisone, prednisolone, prednisone, methylprednisolone) that will not be reduced during the study. Participants will have a minimum total daily dose of >15 mg hydrocortisone (or equivalent).

Compliance, as judged per principal investigator (PI) discretion, with glucocorticoid and mineralocorticoid replacement (if applicable) regimen will be documented during the Screening Period.

A4 and 17-OHP values taken before morning glucocorticoid (GC) dose during Screening Visit 2 should meet the following criteria: A4≥1.5×upper limit of normal (ULN); 17-OHP≥1000 ng/dL (30.3 nmol/L); historical results from within the last 2 years will be accepted if the participant has been on the current dose of GC since that time. Participants who failed screening based on findings the Investigator believes are temporary and not reflective of the usual state of the participant (e.g., normal A4 levels when the participant usually is well above this value) can be considered for rescreening. These cases should be discussed with the Medical Monitor.

Reference ranges for serum A4 values are as follows: about 40-150 ng/dL for adult males; about 30-200 ng/dL for adult females; about 48-140 ng/dL for adolescent males of at least 16 years of age; about 65-210 ng/dL for adolescent males of at least 17 years of age; and about 80-240 ng/dL for adolescent females at least 16 years of age.

If a participant is on estrogen therapy (any route), the dose must be stable for at least 3 months prior to screening.

Female participants who engage in heterosexual intercourse must be of nonchildbearing potential, defined as either surgically sterile (i.e., hysterectomy, bilateral salpingectomy, tubal ligation for at least 3 months, or bilateral oophorectomy), or be postmenopausal with at least 1 year of amenorrhea. In participants with less than 1 year of amenorrhea confirmation of a follicle stimulating hormone (FSH)>30 IU/L is required to confirm menopause. Participants not using hormone replacement therapy must agree to use a highly effective method of contraception from the beginning of Screening until at least 2 weeks after the last dose of study drug. Contraceptive use by men and women should be consistent with local regulations regarding the methods of contraception for those participating in clinical studies. Periodic abstinence (i.e., calendar, ovulation, symptothermal, postovulation methods) and withdrawal are not acceptable methods of contraception.

Male participants should agree to use a condom when sexually active with a female partner of childbearing potential from Screening until at least 2 weeks after the last dose of study drug (or be surgically sterile [i.e., vasectomy with a confirmed absence of sperm in ejaculate]); or agree to remain abstinent on a long term and persistent basis). Male participants should also agree to not donate sperm for the duration of the study and until at least 2 weeks after the last dose of study drug.

### Exclusion Criteria

Participants are excluded from the study if any of the following criteria apply:

Diagnosis of any other form of CAH other than classic 21-hydroxylase deficiency.

Dexamethasone use within 30 days of screening.

History of bilateral adrenalectomy, hypopituitarism, or other condition requiring chronic glucocorticoid therapy.

Night shift workers or any other reason for abnormal sleep/wake cycles.

Clinically significant unstable medical condition or chronic disease other than CAH.

History of major surgery/surgical therapy for any cause within 4 weeks prior to Screening.

Diabetes mellitus treated with insulin for less than 6 weeks prior to Screening, or with change in total daily insulin dose by >15% within 6 weeks prior to Screening.

Poorly controlled diabetes mellitus defined as having a hemoglobin A1c (HbA1c)>8.5% (>69 mmol/mL), or estimated HbA1c based on fructosamine if HbA1c is not evaluable (eg, due to hemoglobinopathies).

Participants with hypothyroidism who are not receiving adequate hormone replacement therapy based on thyroid hormone levels measured at the time of Screening, as determined by the Investigator.

History of unstable angina or acute myocardial infarction within 12 weeks prior to Screening or other clinically significant cardiac disease at the time of Screening as judged by the Investigator.

Concomitant mental condition rendering him/her unable to understand the nature, scope, and possible consequences of the study, and/or evidence of poor compliance with medical instructions.

History of cancer excluding cured/treated dermal squamous or basal cell carcinoma or cervical carcinoma in situ.

Pregnant or lactating.

Known history of illicit drug or alcohol abuse within the last year.

Use of antiandrogen therapy in the past 3 months (e.g., spironolactone, finasteride, cyproterone acetate, flutamide)

Use of testosterone, androgen-containing supplements, aromatase inhibitors, or growth hormone.

Use of medications that are strong inducers of cytochrome P450 (CYP) 3A4 within 30 days prior to Day 1 of the study. These include but are not limited to apalutamide, carbamazepine, enzalutamide, mitotane, phenytoin, rifampin, or St. John's wort.

Use of medications (all routes of administration i.e., oral, topical and inhaled) or ingestion of food that are strong or moderate inhibitors of CYP3A4 within 14 days prior to Day 1 of the study. These include but are not limited to boceprevir, cobicistat, danoprevir and ritonavir, elvitegravir and ritonavir, grapefruit juice (more than 8 ounces daily), indinavir and ritonavir, itraconazole, ketoconazole, lopinavir and ritonavir, nirmatrelvir and ritonavir, paritaprevir and ritonavir and (ombitasvir and/or dasabuvir), posaconazole, ritonavir, saquinavir and ritonavir, telaprevir, tipranavir and ritonavir, telithromycin, troleandomycin, voriconazole, clarithromycin, idelalisib, nefazodone, nelfinavir, aprepitant, ciprofloxacin, conivaptan, crizotinib, cyclosporine, diltiazem, dronedarone, erythromycin, fluconazole, fluvoxamine, imatinib, tofisopam, verapamil.

Use of medications that are strong or moderate inducers of P-gp within 14 days prior to Day 1 of the study. These include but are not limited to apalutamide, carbamazepine, fosphenytoin, lorlatinib, phenytoin, rifampicin, or St. John's wort.

Use of medications that are strong or moderate inhibitors of P-glycoprotein (P-gp) within 14 days prior to Day 1 of the study. These include but are not limited to amiodarone, carvedilol, clarithromycin, dronedarone, itraconazole, lapatinib, lopinavir and ritonavir, propafenone, quinidine, ranolazine, ritonavir, saquinavir and ritonavir, telaprevir, tipranavir and ritonavir, or verapamil.

Use of any investigational drug within the past 60 days or 5 half-lives (whichever is longer) prior to the first dose; or plan to use an investigational drug in another study.

Average (of 3 ECGs) Fridericia's corrected QT (QTcF) interval >450 msec (men) or >470 msec (women), PR interval >220 msec, QRS interval >120 msec, second- or third-degree atrioventricular block, left bundle branch block, or hemiblock at Screening.

Renal insufficiency as measured by estimated glomerular filtration (eGFR) rate <60 mL/min/1.73 m2.

Significant liver disease or alanine aminotransferase (ALT) and/or aspartate aminotransferase (AST)>3×ULN, and/or total bilirubin >1.5×ULN during Screening. Participants with previously diagnosed Gilbert's syndrome not accompanied by other hepatobiliary disorders and associated with total bilirubin <3.5 mg/dL (<51.3 mol/L) will be permitted.

### Study Design

Up to approximately 30 participants will be enrolled in the study. Each cohort will enroll at least 6 participants, with a possible expansion of up to an additional 6 participants in cohorts 1 and 2, to a maximum of 12 evaluable participants in those cohorts. The treatment duration will be up to Day 84 (12 Weeks) per participant. The total study duration will be up to 140 days (up to 4 weeks of screening, 12 weeks of treatment, and 4 weeks of follow-up) per participant. The visit frequency will be at Screening (2 visits), on Day 1 (Baseline), and at Weeks 2 (Day 14), 6 (Day 42), and 12 (Day 84). A Safety Follow-up Visit will occur 2 weeks following the last dose of CRN04894 (Week 14; Day 98), and a telephone call to collect any adverse events (AEs) will occur at 4 weeks after the last dose of CRN04894 (Week 16, Day 112). The study design is discussed in detail below.

During Screening, modification of glucocorticoid doses will not be allowed except for stress (emergency) dosing. Preexisting doses of fludrocortisone will also be maintained throughout the study. After the Screening Period, participants will be assigned to a dose cohort in an open-label fashion. The dose cohorts will run sequentially.

Participants in all cohorts will continue their preexisting glucocorticoid replacement dosing regimens/doses throughout the study. In addition, starting the night of Day 1 and ending the night before the Week 12/end of treatment (EOT) visit, each cohort will administer CRN04894 between 22:00-24:00 (at approximately the same time each night) once daily (QD) before sleep.

Participants in Cohort 1 will receive 80 mg of CRN04894 QD. After at least 3 participants have completed 2 weeks of dosing with 80 mg of CRN04894 QD in Cohort 1, an interim analysis evaluating PD, PK, efficacy, and safety will be reviewed by the Safety Review Committee (SRC). If it is safe to continue, the SRC will recommend dosing of 120 mg of CRN04894 in Cohort 2. However, the SRC can recommend a lower dose (i.e., 40 mg of CRN04894 QD) in Cohort 2 based on review of safety data. The SRC also has the option to recommend increasing enrollment up to a total of 12 participants each in Cohorts 1 and/or 2. In this situation, expanded enrollment in the cohort will be completed, and data from the expanded cohort will be reviewed by the SRC prior to enrollment in the subsequent cohort.

Cohort 1 will first complete enrollment with at least 6 participants prior to commencing Screening for Cohort 2. The same initial dosing and SRC review process as used for the initiation of Cohort 2 would be followed for the optional Cohort 3, in which participants would receive up to 160 mg of CRN04894 QD. The follow-up period will extend from weeks 12 to 16. For all cohorts, safety and PD biomarkers will be obtained at the end of week 14 of the follow-up period. Participants will receive a safety phone call for safety evaluation at the end of the week 16/end of study (EOS).

At any time during the study, the SRC may determine that study drug dosing should be adjusted or halted based on study drug/study stopping criteria.

A participant is considered to have completed the study if they completed the last scheduled procedure specified at the EOS Visit.

A detailed schedule of activities is provided in Table 1.

In Table 1, “X” indicates the activity is scheduled to be performed, and “-” indicates the activity is not scheduled to be performed.

TABLE 1
Schedule of Activities

| | Screening | | | | | | Follow-up | |
|---|---|---|---|---|---|---|---|---|
| | Period | | Treatment Periods | | | | Period | |
| | Visit | | | | | | | |
| | 1 | 2 | 3 | 4 | 5 | 6/EOT | 7 | 8/EOS |
| | Week | | | | | | | |
| | −4 | −2 | 1 | 2 | 6 | 12 | 14 | 16 |
| | Day | | | | | | | |
| | | | | 14 | 42 | 84 | 98 | 112 |
| | −28 to −1 | | 1 | (±2) | (±2) | (±2) | (±3) | (±3) |
| | Onsite Visit | | | | | | | |
| | X | X | X | X | X | X | X | — |
| | Phone Visit | | | | | | | |
| | — | — | — | — | — | — | — | X |
| Informed consent ^{a} | X | — | — | — | — | — | — | — |
| Medical history | X | — | — | — | — | — | — | — |
| Full physical exam, weight, waist | X | — | X | — | — | — | — | — |
| circumference, and vital signs | | | | | | | | |
| Symptom-directed physical examination, | — | — | X | X | X | X | X | — |
| weight, waist circumference and vital signs | | | | | | | | |
| Drug screens | X | X | — | — | — | — | — | — |
| ECG (triplicate) | X | — | X | X | X | X | X | — |
| AE review | X | X | X | X | X | X | X | X |
| Prior or concomitant medications | X | X | X | X | X | X | X | — |
| Adrenal insufficiency awareness training | — | — | X | — | — | — | — | — |
| MRI of adrenal glands | X ^{b} | | | — | — | X | — | — |
| Study drug dispensing ^{c} | — | — | X | X | X | — | — | — |
| Study drug accountability | — | — | — | X | X | X | — | — |
| Glucocorticoid administration diary | — | X | X | X | X | X | X | — |
| Provide diaries with instructions for | X | — | — | — | — | — | — | — |
| glucocorticoid, menstrual, and patient | | | | | | | | |
| severity | | | | | | | | |
| CRN04894 dosing diary | — | — | X | X | X | X | — | — |
| Urinalysis | X | — | — | X | — | X | — | — |
| Hematology, clinical chemistry, INR, | X | — | X | X | X | X | — | — |
| PT/PTT | | | | | | | | |
| Fasting lipid panel and glucose | — | X | X | — | — | X | — | — |
| HbA1C | X | — | — | — | — | X | — | — |
| Free T4, TSH | X | — | — | — | — | X | — | — |
| PD biomarkers | | | | | | | | |
| 17-OHP, ACTH (plasma), | X | X ^{d} | X ^{d} | X ^{d,e} | X ^{d} | X ^{d,e} | X ^{d} | — |
| testosterone, A4, DHEA, DHEA-S, | | | | | | | | |
| 11-DOC, 21-deoxycortisol, 11-OHA4, | | | | | | | | |
| 11-ketoA4, 11-OHT, 11-ketoT | | | | | | | | |
| Aldosterone ^{f} | — | — | X | — | X | X | X | — |
| 21-hydroxylase gene mutation screening | — | — | X | — | — | — | — | — |
| Plasma renin ^{f} | — | — | X | — | X | X | X | — |
| Serology | | | | | | | | |
| HIV antibody, HBsAg, hepatitis C | — | X | — | — | — | — | — | — |
| virus antibody, and COVID antigen | | | | | | | | |
| Sparse PK on site ^{g} | — | — | — | X ^{e} | X | X ^{e} | X | |
| Trough PK | — | — | — | X ^{h} | — | X ^{h} | — | — |
| Pregnancy test (WOCBP) | X | — | X | — | X | X | X | — |
| Menstrual cycle diary (women only) ⁱ | — | — | X | X | X | X | X | — |
| LH/FSH | — | — | X | — | — | X | — | — |
| Sex hormone binding globulin (SHBG) | — | — | X | — | — | X | — | — |
| Estradiol | — | — | X | — | — | X | — | — |
| Patient-reported Severity Items | — | X | X | X | X | X | X | — |
| Questionnaire (Patient-reported CAH | | | | | | | | |
| Severity Items Questionnaire) | | | | | | | | |
| Patient-reported Change Items | — | — | — | — | — | X | — | — |
| Questionnaire | | | | | | | | |
| (Patient-reported CAH Change Items | | | | | | | | |
| Questionnaire) | | | | | | | | |
| Testicular ultrasound (men only) | X ^{b} | | | — | — | X | — | — |
| Treatment Satisfaction Questionnaire | — | — | X | — | — | X | — | — |
| (Treatment Satisfaction Questionnaire For | | | | | | | | |
| Adults and Treatment Satisfaction | | | | | | | | |
| Questionnaire for Patients under 18) | | | | | | | | |
| Adrenal insufficiency assessments | X | X | X | X | X | X | X | — |
| Circadian assessments at selected sites ^{j} | — | — | X | X | — | X | — | — |
| Telephone call to collect AEs | — | — | — | — | — | — | — | X |

11-OHA4 = 11β-hydroxyandrostenedione, 11-DOC = 11-Deoxycorticosterone, 11-ketoT = 11-keto testosterone, 11-OHT = 11-hydroxy testosterone, 17-OHP = 17-hydroxyprogesterone, A4 = androstenedione, ACTH = adrenocorticotropic hormone, AE = adverse event, CAH = congenital adrenal hyperplasia, DHEA = dehydroepiandrosterone, DHEA-S = dehydroepiandrosterone sulfate, ECG = electrocardiogram, EOS = end of study, EOT = end of treatment, FSH = follicle-stimulating hormone; GC = glucocorticoid, HbA1C = hemoglobin A1C, INR = international normalized ratio, PT = prothrombin time, PTT = partial thromboplastin time, TSH = thyroid-stimulating hormone, LH = luteinizing hormone, MRI = Magnetic resonance imaging, P1NP = procollagen type 1 N propeptide, PD = pharmacodynamic, PK = pharmacokinetic, QOL = quality of life, SHBG = sex hormone binding globulin, T4 = thyroxine, VAMS = volumetric absorptive microsampling, WOCBP = women of childbearing potential.
^{a} Informed consent form may be signed up to a week prior to Visit 1
^{b} The MRI and ultrasound should be done after Screening Visit 2 and prior to study drug dosing on Day 1.
^{c} Starting the night of Day 1 and ending the night before the Week 12/EOT visit, each cohort will administer CRN04894 between 22:00-24:00 (at approximately the same time each night) once daily (QD) before sleep.
^{d} For Visits 2-7: Prior to receiving the morning GC replacement dose, a total of 2 blood draws (approximately 1 hour apart) for PD biomarkers will be drawn. The drawing of the PD biomarkers should be one of the first visit activities performed at the start of the visit. Visits should start as early in the morning as possible (preferably 07:00-08:00 am), so as to minimize any delay in administration of the morning GC dose and the timing of each study visit should be as consistent as possible. The second set of PD biomarkers should be drawn no later than 11:00 am. Visit 1 (Week −4) PD biomarker blood draws can be done at any time after the Informed Consent Form is signed and will not be used for Inclusion Criteria assessments. For Screening Visit 2 (Week −2): Blood draw should be done prior to receiving the morning GC dose.
^{e} One blood draw for PK at each visit. At Visit 4 and Visit 6, a Mitra VAMS sample will also be collected at the time of PK collection.
^{f} For participants on fludrocortisone (mineralocorticoid) replacement, serum aldosterone and plasma renin activity will be obtained prior to administration of this medication
^{g} Participants included in the circadian assessments will not have PD biomarkers or PK samples re-drawn on Visits 3, 4, or 6 if the circadian assessment and the visit falls on the same day.
^{h} A Mitra VAMS PK sample will be collected by the study participant before dosing the night before Visit 4 and Visit 6.
ⁱ Completed by the participant every day at home.
^{j} Circadian assessments will be performed at selected sites in settings with 24-hour blood sampling capabilities. Assessments will take place between Day −7 to Day 1, Day 12 to Day 17, and between Day 70 to Day 86. Study drug will be administered at approximately 22:00 during the assessments that occur in the treatment period (between Day 12 to Day 17 and Day 70 to Day 86). Blood will be drawn for measurement of PK, ACTH, A4, 17-OHP, DHEA, 11-DOC, 11-OHA4, 11-ketoA4, 11-OHT, 11-ketoT every 4 hours for a 24-hour period commencing at approximately 10:00 to be followed by samples at 14:00, 18:00, predose 22:00, 02:00, 06:00, and 10:00. Saliva will be collected simultaneously with blood for measurement of A4, 17-OHP, 11-OHA4, 11-ketoA4, 11-OHT, 11-ketoT, and testosterone except at 02:00 (to allow the participant to sleep). In addition, blood will be drawn for testosterone, DHEA-S, and 21-deoxycortisol once at 06:00 am.

### Genetics

Mutations in the CYP21A2 gene cause varying degrees of loss of 21-hydroxylase activity, resulting in different phenotypes. Participants will be screened for 21-hydroxylase gene mutation at the start of the treatment period.

### Pharmacokinetics (PK)

During the treatment period, for participants who are not participating the 24-hour inpatient circadian rhythm assessment, blood samples will be collected for the measurement of CRN04894 concentration in plasma and whole blood (using Mitra volumetric absorptive microsampling [VAMS]). The actual date and time (24-hour clock time) of collection of each sample will be recorded. The actual date and time of last dose of study drug prior to each sample collection will also be recorded.

CRN04894 concentrations in plasma and whole blood will be listed and summarized by cohort and study day for the PK analysis population at each scheduled collection time point with descriptive statistics.

### Pharmacodynamics (PD)

A pharmacodynamic biomarker panel will be drawn. The biomarker panel will include the following:
17-hydroxyprogesterone (17-OHP);
Adrenocorticotropic hormone (ACTH) (plasma);
Testosterone;
Androstenedione (A4);
Dehydroepiandrosterone (DHEA);
Dehydroepiandrosterone sulfate (DHEA-S);
11-deoxycorticosterone (11-DOC);
21-deoxycortisol;
11β-hydroxyandrostenedione (11-OHA4);
11-ketoandrostenedione (11-ketoA4);
11β-hydroxytestosterone (11-OHT);
11-ketotestosterone (11-ketoT);
Aldosterone; and
Renin.

A PD biomarker draw will be taken at the beginning of the treatment period prior to the first dosing of CRN4894 to establish a PD biomarker baseline.

### Efficacy Assessments

Magnetic resonance imaging: Adrenal magnetic resonance imaging (MRI) should be performed locally. Image acquisition standards will be provided and must be followed to allow proper evaluation of adrenal volume. All MRI assessments will be assessed by the Investigator for any findings that require immediate medical attention and will also be read by an MRI central radiologist. The clinical significance of any MRI findings will be determined by the Investigator, including after the central reading result is available.

Ultrasound: Testicular ultrasound will be performed according to the study site procedures for the evaluation of testicular adrenal rest tumors (TARTs). Two ultrasounds are required for all male participants; 1) after screening but before treatment and 2) at the end of treatment. The results will be recorded. All ultrasound assessments will be assessed by the Investigator for any findings that require immediate medical attention and will also be read by an ultrasound central radiologist. The clinical significance of any ultrasound findings will be determined by the Investigator, including after the central reading result is available.

Pharmacodynamics (PD) biomarker draws:

Screening Visit 1: PD biomarker blood draws can be done at any time after the Informed Consent Form is signed and will not be used for Inclusion Criteria assessments.

Screening Visit 2: Blood draw should be done prior to receiving the morning GC dose.

Visits 2-7: Prior to receiving the morning GC replacement dose, a total of 2 blood draws (approximately 1 hour apart) for PD biomarkers will be drawn. The drawing of the PD biomarkers should be one of the first visit activities performed at the start of the visit. Visits should start as early in the morning as possible (preferably 07:00-08:00), so as to minimize any delay in administration of the morning GC dose and the timing of each study visit should be as consistent as possible. The second set of PD biomarkers should be drawn no later than 11:00 am.

Circadian rhythm assessment: At specified visits, participants will be admitted to a metabolic unit for 24-hour Circadian Rhythm Assessments. Circadian assessments will be performed at selected sites in settings with 24-hour blood sampling capabilities. Assessments will take place between Day −7 to Day 1, Day 12 to Day 17, and between Day 70 to Day 86. Study drug will be administered at approximately 22:00 during the study treatment (between Day 12 to Day 17, and Day 70 to Day 86). Blood will be drawn for measurement of PK, ACTH, A4, 17-OHP, DHEA, 11-DOC, 11-OHA4, 11-ketoA4, 11-OHT, and 11-ketoT every 4 hours for a 24-hour period commencing at approximately 10:00, to be followed by samples at 14:00, 18:00, predose 22:00, 02:00, 06:00, and 10:00. Saliva will be collected simultaneously with blood for measurement of A4, 17-OHP, 11-OHA4, 11-ketoA4, 11-OHT, 11-ketoT, and testosterone except at 02:00 (to allow the participant to sleep). In addition, blood will be drawn for testosterone, DHEA-S, and 21-deoxycortisol once at 06:00 am.

### Safety Assessments

Physical examinations: A full physical examination will be performed, and body weight and waist circumference will be measured at the time points specified in the schedule of activities. Height and body mass index (BMI) are to be measured once, at Screening. Examination is to include assessment of head (external), eyes, ears, nose, and throat, lungs, cardiovascular system, abdomen, musculoskeletal system, skin, lymph nodes, central nervous system (CNS), and, where appropriate, other body systems.

A symptom-directed physical examination due to an AE or other available safety data may be conducted as an unscheduled assessment.

Vital signs: Vital signs will be measured (resting and standing blood pressure, resting respiratory rate, and resting body temperature) as per standard practice.

Standard blood pressure should be measured in the supine position after resting for 5 minutes. Measure standing blood pressure and standing pulse rate after the participant has been supine for 5 minutes followed by standing for 2 to 5 minutes.

Electrocardiograms (ECGs): ECGs will be collected at the time points specified in the schedule of activities. The ECG assessment is to be a standard 12-lead ECG performed in triplicate (1 to 3 minutes apart) after the participant has rested quietly in the supine position for at least 10 minutes without significant stimulation (noise, television, etc.). Additional ECGs outside of planned assessments are to be collected, per protocol, if clinically indicated (eg, participants with palpitations, lightheadedness).

The ECG parameters that are to be assessed include a summary of findings as well as measurement of the pulse rate, time interval between Q and T wave (QT), corrected QT by the Fridericia formula (QTcF), and time interval between P and R waves (PR), and time interval of the QRS complex (QRS duration) based on the ECG machine readings.

All ECG assessments will be initially assessed by the Investigator for any findings that require immediate medical attention and will also be read by an ECG central reader. The clinical significance of any ECG findings will be determined by the Investigator, including after the central reading result is available. Any potentially significant outlier values should be confirmed by the central ECG reader. Any ECG measurement determined to be clinically significant (occurring after signing the ICF) will be noted as an AE. Such abnormalities will be monitored until the end of the study or until resolution if considered related to the study drug.

Clinical safety laboratory tests: The clinical laboratory tests, and their timing and frequency, are disclosed in the schedule of activities. Safety laboratory tests are to be performed by the central laboratory. Reference ranges will be per the central laboratory. The Investigator must review the laboratory report, document this review, and record any clinically significant changes occurring during the study as AEs. The laboratory reports must be filed with the source documents. All laboratory tests with values considered clinically significantly abnormal during participation in the study should be repeated until the values return to normal or baseline or are no longer considered clinically significant by the Investigator or medical monitor. If such values do not return to normal/baseline within a period of time judged reasonable by the Investigator, the etiology should be identified, and the Sponsor notified.

Clinical safety laboratory tests include the following: blood urea nitrogen (BUN); potassium; creatinine; sodium; calcium; random glucose; glucose (fasting); fasting lipid panel; aspartate aminotransferase/serum glutamic-oxaloacetic transaminase (AST/SGOT); alanine aminotransferase/serum glutamic-pyruvic transaminase (ALT/SGPT); alkaline phosphate; total and direct bilirubin; total protein; uric acid; chloride; phosphate; magnesium; albumin; indirect bilirubin; amylase; lipase.

Protocol-required laboratory tests are summarized in Table 2:

TABLE 2
Protocol Required Laboratory Tests

| Laboratory Tests | Parameters | | |
|---|---|---|---|
| Hematology | Platelet count | | |
| | HbA1C | | |
| | Red blood cell (RBC) count/Hemoglobin /Hematocrit | | |
| | RBC indices: | MCV | |
| | | MCH | |
| | | % Reticulocytes | |
| | White blood cell (WBC) | Neutrophils | |
| | count with differential: | Lymphocytes | |
| | | Monocytes | |
| | | Eosinophils | |
| | | Basophils | |
| Hematology | PT/INR | PTT | |
| (coagulation) | | | |
| Clinical chemistry ^{a} | BUN | AST/SGOT | Phosphate |
| | Potassium | ALT/SGPT | Magnesium |
| | Creatinine | Alkaline | Albumin |
| | Sodium | phosphatase ^{b} | Indirect bilirubin |
| | Calcium | Total and direct | Amylase |
| | Random glucose | bilirubin | Lipase |
| | Glucose (fasting) | Total protein | |
| | Fasting lipid panel | Uric acid | |
| | | Chloride | |
| Hormones | Free T4 | SHBG | Aldosterone |
| | TSH | Estradiol | Renin |
| | LH/FSH | | |
| PD biomarkers | 17-OHP | 21-deoxycortisol | |
| | ACTH (plasma) | 11-OHA4 | |
| | Testosterone | 11-ketoA4 | |
| | A4 | 11-OHT | |
| | DHEA | 11-ketoT | |
| | DHEA-S | | |
| | 11-DOC | | |
| PK | PK parameters | | |
| 21-hydroxylase gene | Gene mutation analysis | | |
| mutation screening | | | |
| Circadian rhythm | PK | 11-DOC | Drawn once at 06:00: |
| assessments (blood | ACTH (plasma) | 11-OHA4 | Testosterone |
| and saliva) collected | A4 | 11-ketoA4 | DHEA-S |
| every 4 hours for | 17-OHP | 11-OHT | 21-Deoxycortisol |
| 24 hours | DHEA | 11-ketoT | |

| | |
|---|---|
| Routine urinalysis | pH, glucose, protein, blood, ketones, bilirubin, urobilinogen, nitrite, leukocyte |
| | esterase by dipstick |
| | Microscopic examination (if blood or protein is abnormal) |
| Pregnancy testing | Highly-sensitive serum or urine hCG pregnancy test (as needed for women of |
| | childbearing potential) ^{c} |
| | Follicle-stimulating hormone and estradiol (in women of nonchildbearing potential |
| | only) |
| Toxicology | Serum or urine alcohol and drug screen (to include at minimum: amphetamines, |
| | barbiturates, cocaine, and opiates) |
| Serology | HIV antibody, HBsAg, hepatitis C virus antibody, and COVID antigen |

11-OHA4=11βhydroxyandrostenedione; 11-DOC=11-Deoxycorticosterone; 11-ketoT=11-keto testosterone; 11-OHT=11-hydroxy testosterone; 17-OHP=17-hydroxyprogesterone; A4=androstenedione; ACTH=adrenocorticotropic hormone; ALT=alanine aminotransferase; AST=aspartate aminotransferase; BUN=blood urea nitrogen; COVID=coronavirus disease; DHEA=dehydroepiandrosterone; DHEA-S=dehydroepiandrosterone sulfate; ECG=electrocardiogram; EOS=end of study; FSH=follicle-stimulating hormone; HbA1C=hemoglobin A1C; HBsAg=hepatitis B surface antigen; HCG=human chorionic gonadotropin; HIV=human immunodeficiency virus; INR=international normalized ratio; LH=luteinizing hormone; MCH=mean corpuscular hemoglobin; MCV=mean corpuscular volume; PK=pharmacokinetic; PT=prothrombin time; PTT=partial thromboplastin time; RBC=red blood cell; SGOT=serum glutamic-oxaloacetic transaminase; SGPT=serum glutamic-pyruvic transaminase; SHBG=sex hormone binding globulin; T4=thyroxine; TSH=thyroid-stimulating hormone; WOCBP=women of child bearing potential.
^{a}Analyses of liver chemistry abnormalities as evidenced by liver tests and discontinuation based on evidence of liver injury will be performed and must be reported to Sponsor in an expedited manner (excluding studies of hepatic impairment or cirrhosis).
^{b}If alkaline phosphatase is elevated, consider fractionating.
^{c}Local urine testing will be standard for the protocol unless serum testing is required by local regulation or IRB/IEC.

Pregnancy testing: Pregnancy testing (urine or serum as required by local regulations) should be conducted as per the schedule of activities. Pregnancy testing (urine or serum as required by local regulations) should be conducted at the end of relevant systemic exposure plus an additional 14 days and correspond with the time frame for female participant contraception. Additional serum or urine pregnancy tests may be performed, as determined necessary by the investigator or required by local regulation, to establish the absence of pregnancy at any time during the participant's participation in the study.

### Adverse Events (AE), Serious Adverse Events (SAE), and Other Safety Reporting

An AE is the development of an untoward medical condition or the deterioration of a preexisting medical condition following or during exposure to a pharmaceutical product, whether or not considered causally related to the product and occurring after the moment of signing the informed consent form until the final follow-up contact.

An AE can therefore be any unfavorable and unintended sign (including an abnormal laboratory finding), symptom, or disease (new or exacerbated) during a clinical study, even if no study drug has been administered.

Treatment-emergent adverse events (TEAEs) are AEs occurring or worsening after administration of study drug.

The investigator and any qualified designees are responsible for detecting, documenting, and recording events that meet the definition of an AE or SAE and remain responsible for following up on all AEs or SAEs whether or not considered related to the study drug or study procedures, or that caused the participant to discontinue the CRN04894. This includes events reported by the participant (or, when appropriate, by a caregiver, surrogate, or the participant's legally authorized representative).

Time period and frequency for collecting AE and SAE information: All AEs and SAEs will be collected starting from the signing of the informed consent form until 4 weeks after the last dose of study drug. When an AE/SAE occurs, it is the responsibility of the Investigator to collect and review all documentation (e.g., hospital progress notes, laboratory reports, and diagnostics reports) related to the event. The Investigator is to then record all relevant AE/SAE information in the eCRF and report all SAEs on the paper SAE Report Form. All SAEs will be recorded and reported to the Sponsor or designee immediately and under no circumstance should this exceed 24 hours of first awareness. The investigator will submit any updated SAE data to the Sponsor within 24 hours of it being available. Investigators are not obligated to actively seek information on AEs or SAEs after conclusion of the study participation. However, if the investigator learns of any SAE, including a death, at any time after a participant has been discharged from the study, and the investigator considers the event to be reasonably related to the study drug or study participation, the investigator must promptly notify the Sponsor.

Time Period and Frequency for Collecting AE and SAE information: All AEs and SAEs will be collected starting from the signing of the ICF until 4 weeks after the last dose of study drug. When an AE/SAE occurs, it is the responsibility of the Investigator to collect and review all documentation (eg, hospital progress notes, laboratory reports, and diagnostics reports) related to the event.

The Investigator is to then record all relevant AE/SAE information in the eCRF and report all SAEs on the paper SAE Report Form.

All SAEs will be recorded and reported to the Sponsor or designee immediately and under no circumstance should this exceed 24 hours of first awareness. The investigator will submit any updated SAE data to the Sponsor within 24 hours of it being available.

Investigators are not obligated to actively seek information on AEs or SAEs after conclusion of the study participation. However, if the investigator learns of any SAE, including a death, at any time after a participant has been discharged from the study, and the investigator considers the event to be reasonably related to the study drug or study participation, the investigator must promptly notify the Sponsor.

Method of detecting AEs and SAEs: Care will be taken not to introduce bias when detecting AEs and/or SAEs. Open-ended and nonleading verbal questioning of the participant is the preferred method to inquire about AE occurrences.

Follow-up of AEs and SAEs: After the initial AE/SAE report, the investigator is required to proactively follow each participant at subsequent visits/contacts. All SAEs and AEs of special interest will be followed until resolution, stabilization, the event is otherwise explained, or the participant is lost to follow-up.

The Investigator is obligated to perform or arrange for the conduct of supplemental measurements and/or evaluations as medically indicated or as requested by the Sponsor or designee to elucidate the nature and/or causality of the AE or SAE as fully as possible. This may include additional laboratory tests or investigations, histopathological examinations, or consultation with other health care professionals.

If the participant dies during participation in the study or during a recognized follow-up period, the Investigator is to provide the Sponsor or designee with a copy of any postmortem findings including histopathology, to the extent practical.

Regulatory Reporting Requirements for SAEs: Prompt notification by the investigator to the Sponsor of an SAE is essential so that legal obligations and ethical responsibilities towards the safety of participants and the safety of a study drug under clinical investigation are met.

The Sponsor has a legal responsibility to notify both the local regulatory authority and other regulatory agencies about the safety of a study drug under clinical investigation. The Sponsor will comply with country-specific regulatory requirements relating to safety reporting to the regulatory authority, institutional review boards (TRBs)/independent ethics committees (IECs), and investigators.

An investigator who receives an investigator safety report describing an SAE or other specific safety information (eg, summary or listing of SAEs) from the Sponsor will review and then file it along with the Investigator's Brochure (IB)/package insert or other documents and will notify the IRB/IEC, if appropriate according to local requirements.

Investigator safety reports must be prepared for suspected unexpected serious adverse reactions (SUSARs) according to local regulatory requirements and Sponsor policy and forwarded to investigators, as necessary.

Pregnancy: Details of all pregnancies in female participants and, if indicated, female partners of male participants will be collected after the start of study drug and until time period for reporting pregnancies should align with the time period for postintervention contraception.

While pregnancy itself is not considered to be an AE or SAE, any pregnancy complication or elective termination of a pregnancy for medical reasons will be reported as an AE or SAE.

Abnormal pregnancy outcomes (eg, spontaneous abortion, fetal death, stillbirth, congenital anomalies, ectopic pregnancy) are considered SAEs and will be reported as such.

The participant/pregnant female partner will be followed to determine the outcome of the pregnancy. The investigator will collect follow-up information on the participant/pregnant female partner and the neonate, and the information will be forwarded to the Sponsor.

Any poststudy pregnancy-related SAE considered reasonably related to the study drug by the investigator will be reported to the Sponsor. While the investigator is not obligated to actively seek this information in former study participants/pregnant female partners, he or she may learn of an SAE through spontaneous reporting.

Any female participant who becomes pregnant while participating in the study will discontinue study drug or be withdrawn from the study.

Adverse Events of Special Interest (AESI): An adverse event of special interest (AESI) is an AE of special medical or scientific interest to the Sponsor. For this study, the following event is considered to be an AESI: adrenal insufficiency.

### Statistical Considerations

General Considerations: Descriptive statistics, including number of nonmissing observations, arithmetic mean, standard deviation, median, first and third quartiles, minimum, and maximum will be used to summarize continuous endpoints. The number of participants and percentages in each level will be used to summarize categorical or ordinal endpoints. Unless specified otherwise, missing or unreported categorical results will be included in the summary and the number of participants in the Full Analysis Set will be used as denominator.

Safety Analysis: Safety endpoints include study drug exposure, adverse events, clinical laboratories, vital signs and 12-lead ECGs. Unless specified otherwise safety endpoints will be summarized by planned dose level for each cohort and overall.

Drug Exposure: CRN04894 treatment exposure will be summarized by the planned dose level for each cohort. Exposure summaries will include number of doses, duration of treatment, and actual dose received.

Adverse Events: The adverse events will be summarized by the planned dose level for each cohort and overall. AEs will be coded according to the Medical Dictionary for Regulatory Activities (MedDRA) version 25.0 or later.

TEAEs are defined as AEs that occur or worsen at the time of or following the administration of the first dose of study drug. If the time of the AE is not available, then TEAEs will be those occurring on or after the first day of the administration of study drug. Adverse events that are not treatment-emergent will not be included in the summary but will be included in the listing.

A summary table of AEs will be presented detailing the number and percentage of participants, and number of events for the following categories:
At least one TEAE;
At least one study drug-related TEAE;
At least one severe TEAE;
At least one serious TEAE;
At least one study drug-related serious TEAE;
At least one TEAE leading to treatment discontinuation.

Treatment-related AEs are defined as those AEs with relationship to study drug as “Related.” Any AE with a missing relationship will be considered as related.

The incidence of TEAEs will be presented using counts, percentage, and number of events by System Organ Class (SOC) and preferred term (PT). SOCs will be sorted in descending frequency and the PTs within each SOC will be sorted in descending frequency based on the percentage of AEs for all participants. A participant who has multiple occurrences of an event associated with a particular SOC or a PT within a SOC, will only be counted once for that SOC and/or PT. Separate summaries for each of the categories in the overall AE summary table will be created by SOC and PT.

In addition to the AE summaries by SOC and PT, the following will be presented by descending frequency of PT based on the percentage of AE for all participants:
TEAEs;
Treatment-related TEAEs; and
Severe TEAEs.

The following additional listings will be provided:
Listing of SAEs;
Listing of TEAEs leading to treatment discontinuation;
Listing of Severe TEAEs; and
Listing of deaths.

Clinical Laboratory Tests: All continuous laboratory parameters in hematology and clinical chemistry will be summarized descriptively at each clinical visit, together with the corresponding changes from baseline. Other continuous clinical laboratory tests will be summarized in the same way. The number and percentage of participants outside of the normal range (normal, low, and high) will also be summarized by visit.

Time profile of mean and standard error of change from baseline of laboratory tests of interest will be plotted.

All of the laboratory results will be listed including both scheduled and unscheduled results and results outside of the normal reference will be noted on the listing.

Vital Signs, Weight, and Waist Circumference: Vital signs include resting blood pressure (resting and standing), resting respiratory rate, and resting temperature. Vital signs, weight, and waist circumference will be summarized descriptively at each clinical visit. Time profile of mean and standard error of change from baseline of waist circumference will be plotted.

All vital signs, weight, and waist circumference measurement will be included in the listings including both scheduled and unscheduled results.

Electrocardiogram: Standard 12-Lead ECG data will be collected based on the schedule in schedule of activities. For all parameters (heart rate, QT, QTcF, PR intervals, and QRS) results at each visit and change from baseline will be presented. The evaluation results will be summarized at each visit as well. The mean of the triplicates will be used for summary purposes.

All ECG measurements with the original triplicates results and evaluation results including both scheduled and unscheduled results will be listed.

Primary Efficacy Analysis: The primary efficacy endpoint is defined as the change from baseline in early morning (before 11:00) serum A4 at Week 12. Analysis of Covariance (ANCOVA) will be used to analyze change from baseline in A4. The model will include baseline value as a covariate, and sex and dose cohort as fixed effects. Sensitivity analyses such as modelling the percent change from baseline, inclusion of additional baseline characteristics in the ANCOVA model, and nonparametric test might be carried out, and the details will be specified in the SAP. The change from baseline at each postbaseline visit will be presented graphically with the box plots. The descriptive statistics will be presented by the planned dose level in each cohort and overall for A4 at each visit and corresponding change from baseline.

Sample Size Determination: The sample size is based on practical considerations. No formal sample size and power calculation will be carried out for the efficacy endpoints in the study. Up to approximately 30 participants will be enrolled in the study. Each cohort will enroll at least 6 participants, with a possible expansion of up to an additional 6 participants in any single cohort, to a maximum of 12 evaluable participants in that cohort. Interim Analysis: For either Cohort 1 or Cohort 2, after 3 or more participants in the given Cohort have completed 2 weeks of dosing, an analysis will be prepared for SRC purpose including PD, PK, efficacy, and safety endpoints. After all participants in Cohort 2 have gone through the treatment period or discontinued study treatment early, an analysis of the efficacy and safety endpoints of the two cohorts are planned to support regulatory interactions and decision-making.

### Cohort 1 Initial Analysis

The initial analysis summarizes the data of four participates (n=4) of Cohort 1 who received 80 mg of CRN04894 QD. There were three female/women participants (n=3) and one male/men participant (n=1).

Demographics and baseline characteristics of participants of Cohort 1 are presented in Table 3 below:

TABLE 3
Cohort 1 Initial Analysis, Demographics and Baselines

| Age (years), Mean (Range) | 33.8 | (25-42) |
|---|---|---|
| Female, n (%) | 3 | (75%) |
| BMI (kg/m²), Mean (SD) | 29.7 | (5.0) |
| Time Since CAH Diagnosis (years), Mean [symbol] | 34.4 | (0.03) |
| Morning Serum A4, Mean (SD) | ng/dL: 331 (249) | |
| | nmol/L: 11.6 (8.7) | |
| Morning Serum 17-OHP, Mean (SD) | ng/dL: 5795 (915) | |
| | nmol/L: 176 (28) | |
| Morning testosterone female/women, Mean (SD) | ng/dL: 114 (104) | |
| | nmol/L: 3.9 (3.6) | |
| Morning testosterone male/men, Mean (SD) | ng/dL: 389 (NA) | |
| | nmol/L: 13.5 (NA) | |
| Morning A4/testosterone male/men, Mean (SD) | 0.3 | (NA) |
| Morning Plasma ACTH, Mean (SD) | pg/mL: 545 (408) | |
| | pmol/L: 120 (90) | |
| Morning Serum 21-Deoxycortisol, Mean (SD) | ng/dL: 1675 (2006) | |
| | nmol/L: 48 (58) | |
| Mean Baseline Glucocorticoid (GC) Dose* | 36 mg/day | |
| | (20.2 mg/m²/day) | |
| Baseline GC Dose Group*, n (%) (<12 | 0 | |
| mg/m²/day) | | |
| Baseline GC Dose Group*, n (%) (≥12 | 4 | (100%) |

mg/m²/day)

[symbol]

= 2
*In hydrocortisone equivalents

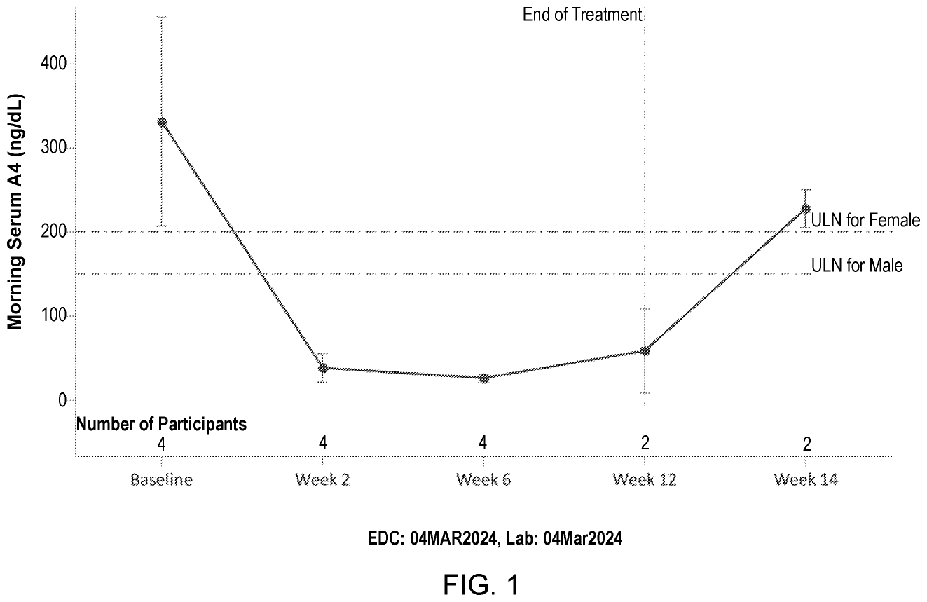

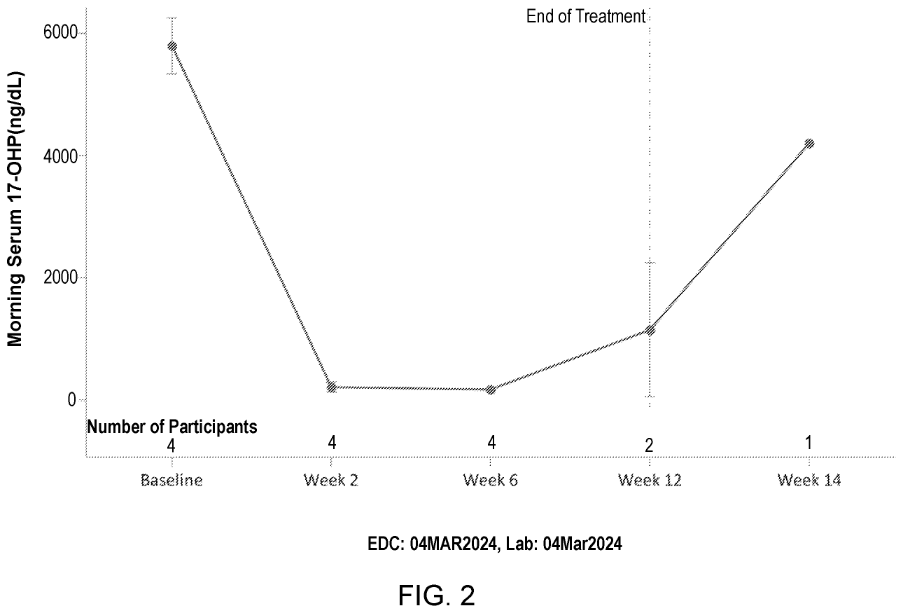

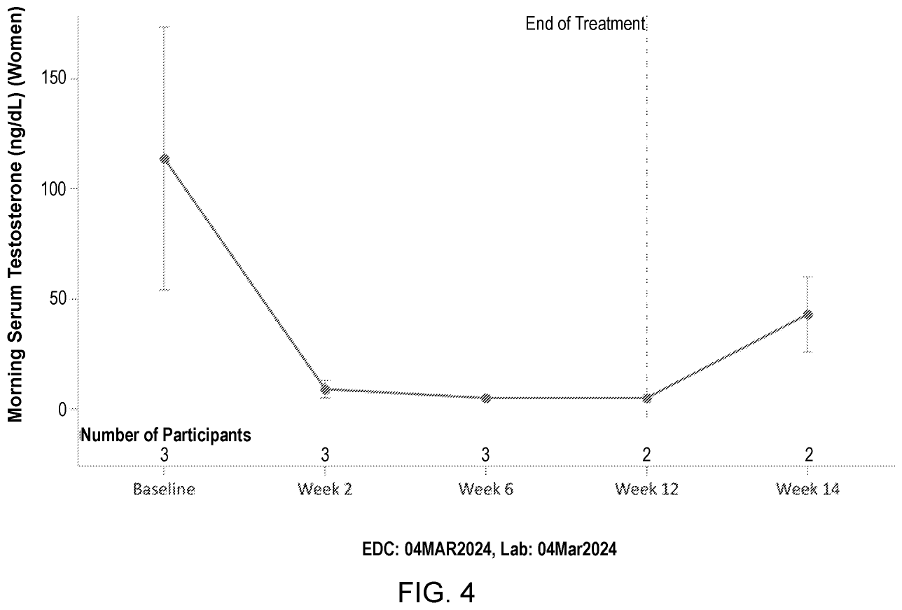

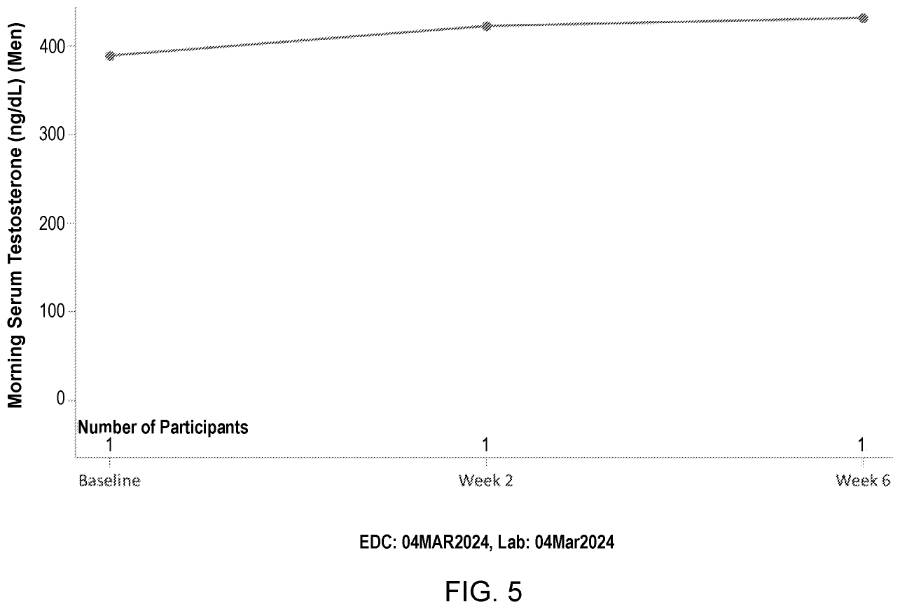

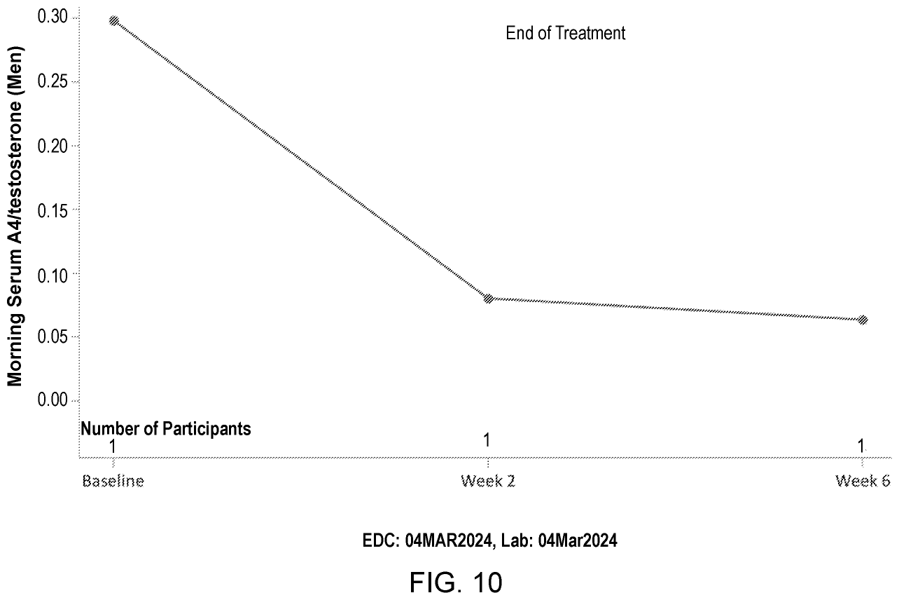

Cohort 1 initial analysis of % changes in values from baseline of morning serum A4, morning serum 17-OHP, morning serum testosterone in females, morning serum testosterone in males, and morning serum A4/testosterone in males are presented in Table 4 below. These changes from baseline are also depicted in FIG. 1, FIG. 2, FIG. 4, FIG. 5, and FIG. 10, respectively.

TABLE 4
Cohort 1 Initial Analysis, % Change in Value

| | | Week | Week | Week |
|---|---|---|---|---|
| Value | Mean/Median | 2 | 6 | 12 |
| % Change in morning serum A4 | Mean | −86 | −90 | −88 |
| | Median | −88 | −94 | −88 |
| % Change in morning 17-OHP | Mean | −96 | −97 | −83 |
| | Median | −96 | −97 | −83 |
| % Change in morning | Mean | −61 | −65 | −97 |
| testosterone (female/women) | Median | −86 | −96 | −97 |
| % Change in morning | Mean | 9 | 11 | — |
| testosterone (male/men) | Median | 9 | 11 | — |
| % Change in morning | Mean | −73 | −79 | — |
| A4/testosterone (male/men) | Median | −73 | −79 | — |

### Initial Cohort 1 Analysis Discussion

Patients (aged ≥18 to 75, ≥16 years in USA) with classical CAH on a stable dose of glucocorticoid replacement for at least six months were enrolled and received 80 mg once daily, oral CRN04894 for 12 weeks followed by a four-week washout. Four participants (three females, median age 34 (range 25-42)) have completed six weeks and two participants have completed 12 weeks of therapy.

Baseline morning A4 levels in these participants ranged from 116 to 604 ng/dL (normal reference ranges are 30-200 ng/dL for females and 40-150 ng/dL for males). In all participants, administration of CRN04894 resulted in rapid and profound A4 reduction within two weeks, which were maintained for the duration of therapy: reductions (mean±SD) of 86%±10% (n=4); 90%±9% (n=4); and 88%±15% (n=2) at Weeks 2, 6 and 12, respectively with all A4 levels ending below the upper limit of normal (<ULN) at all visits following treatment.

Baseline 17-OHP levels in these participants ranged from 4740 to 6905 ng/dL (normal reference ranges are <220 ng/dL for males and for females <80 ng/dL [follicular], <285 ng/dL [luteal]). In all participants, administration of CRN04894 resulted in rapid and profound 17-OHP reduction within two weeks, which were maintained for the duration of therapy: reductions of 96%±2% (mean±SD) (n=4); 97%±0.3% (n=4); & 83%±22% (n=2) at Weeks 2, 6, and 12, respectively.

In the three female participants, baseline morning testosterone was 114±±104 ng/dL (mean±SD, RR 8-60 ng/dL) with reductions of 61%±53% (mean±SD) (n=3); 65%±56% (n=3), & 97%±1% (n=2) at Weeks 2, 6, and 12, respectively. At baseline, the A4/T ratio in the male patient was 0.3 (normal <0.2), and at subsequent visits was normal at 0.08 (week 2) and 0.06 (week 6). Two female participants in this cohort menstruated for the first time in over 2 years while receiving CRN04894.

Mean baseline morning ACTH ranged from 155 to 1009 pg/mL (normal reference range is 7.2-63 pg/mL). Modest variations in ACTH levels without a consistent directional trend were observed during treatment: −25%±57% (n=4); 51%±87% (n=4); & −13%±67% (n=2) at Weeks 2, 6, and 12, respectively.

1. A method of treating congenital adrenal hyperplasia (CAH) in a subject comprising administering to the subject a compound of Formula (I), or a pharmaceutically acceptable salt thereof:

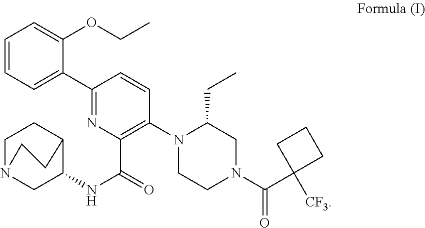 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

2. A method of treating hyperandrogenism in a subject with CAH comprising administering to the subject a compound of Formula (I), or a pharmaceutically acceptable salt thereof:

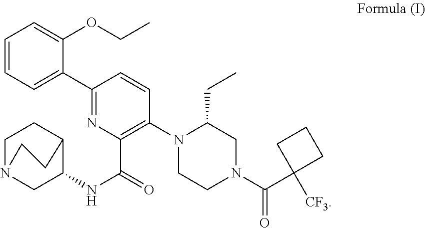 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

3. The method of any one of claims 1-2, wherein the compound of Formula (I), or the pharmaceutically acceptable salt thereof, is administered in a therapeutically effective amount.

4. The method of any one of claims 1-3, wherein the compound of Formula (I), or the pharmaceutically acceptable salt thereof, is administered in an amount equivalent to about 40 mg to about 160 mg of Formula (I).

5. The method of any one of claims 1-3, wherein the compound of Formula (I), or the pharmaceutically acceptable salt thereof, is administered in an amount equivalent to about 80 mg to about 160 mg of Formula (I).

6. The method of any one of claims 1-3, wherein the compound of Formula (I), or the pharmaceutically acceptable salt thereof, is administered in an amount equivalent to about 80 mg to about 120 mg of Formula (I).

7. The method of any one of claims 1-3, wherein the compound of Formula (I), or the pharmaceutically acceptable salt thereof, is administered in an amount equivalent to about 40 mg of Formula (I).

8. The method of any one of claims 1-3, wherein the compound of Formula (I), or the pharmaceutically acceptable salt thereof, is administered in an amount equivalent to about 80 mg of Formula (I).

9. The method of any one of claims 1-3, wherein the compound of Formula (I), or the pharmaceutically acceptable salt thereof, is administered in an amount equivalent to about 120 mg of Formula (I).

10. The method of any one of claims 1-3, wherein the compound of Formula (I), or the pharmaceutically acceptable salt thereof, is administered in an amount equivalent to about 160 mg of Formula (I).

11. The method of any one of claims 1-10, wherein the compound of Formula (I), or the pharmaceutically acceptable salt thereof, is administered in one or more unit dosage forms.

12. The method of claim 11, wherein the unit dosage form comprises the compound of Formula (I), or the pharmaceutically acceptable salt thereof, in an amount of equivalent to about 40 mg of Formula (I).

13. The method of any one of claims 11-12, wherein the unit dosage form is an oral unit dosage form.

14. The method of any one of claims 1-13, wherein the compound of Formula (I), or the pharmaceutically acceptable salt thereof, is administered daily.

15. The method of any one of claims 1-13, wherein the compound of Formula (I), or the pharmaceutically acceptable salt thereof, is administered once daily.

16. The method of any one of claims 1-15, wherein the CAH is caused by 21-hydroxylase deficiency.

17. The method of any one of claims 1-16 wherein the subject is concurrently on glucocorticoid replacement therapy.

18. The method of any one of claims 1-16, wherein the subject is concurrently on hydrocortisone, prednisolone, prednisone, or methylprednisolone therapy.

19. The method of any one of claims 1-16, wherein the subject is concurrently on mineralocorticoid replacement therapy.

20. The method of any one of claims 1-16, wherein the subject is concurrently on fludrocortisone therapy.

21. The method of any one of claims 1-20, wherein treating comprises reducing the subject's serum levels of androstenedione (A4) from baseline.

22. The method of claim 21, wherein the subject's serum levels of A4 are reduced by >75% from baseline.

23. The method of claim 21, wherein the subject's serum levels of A4 are reduced by >80% from baseline.

24. The method of claim 21, wherein the subject's serum levels of A4 are reduced by >85% from baseline.

25. The method of claim 21, wherein the subject's serum levels of A4 are reduced by >90% from baseline.

26. The method of any one of claims 1-20, wherein treating comprises reducing the subject's serum levels of 17-hydroxyprogesterone (17-OHP) from baseline.

27. The method of claim 26, wherein the subject's serum levels of 17-OHP are reduced by >75% from baseline.

28. The method of claim 26, wherein the subject's serum levels of 17-OHP are reduced by >80% from baseline.

29. The method of claim 26, wherein the subject's serum levels of 17-OHP are reduced by >85% from baseline.

30. The method of claim 26, wherein the subject's serum levels of 17-OHP are reduced by >90% from baseline.

31. The method of claim 26, wherein the subject's serum levels of 17-OHP are reduced by >95% from baseline.

32. The method of any one of claims 1-20, wherein treating comprises reducing the subject's serum levels of testosterone from baseline.

33. The method of claim 32, wherein the subject's serum levels of testosterone are reduced by >50% from baseline.

34. The method of claim 32, wherein the subject's serum levels of testosterone are reduced by >60% from baseline.

35. The method of claim 32, wherein the subject's serum levels of testosterone are reduced by >70% from baseline.

36. The method of claim 32, wherein the subject's serum levels of testosterone are reduced by >80% from baseline.

37. The method of any one of claims 32-36, wherein the subject is a female.

38. The method of any one of claims 1-37, wherein treating comprises reducing the subject's serum levels of one or more of 11-deoxycorticosterone (11-DOC), 11β-hydroxyandrostenedione (11-OHA4), 11-ketoandrostenedione (11-ketoA4), 11β-hydroxytestosterone (11-OHT), and 11-ketotestosterone (11-ketoT) from baseline.

39. The method of any one of claims 21-36, wherein the baseline is measured in the morning.

40. The method of any one of claims 1-39, wherein treating comprises reducing adrenal gland size.

41. The method of any one of claims 1-39, wherein treating comprises reducing waist circumference.

42. The method of any one of claims 1-39, wherein treating comprises reducing levels of hemoglobin A1C (HbA1C).

43. The method of any one of claims 1 and 3-39, wherein the subject has hyperandrogenism.

44. The method of any one of claims 1-39, wherein the subject has acne.

45. The method of claim 44, wherein the subject reports their acne has improved following administration of the compound of Formula (I), or a pharmaceutically acceptable salt thereof.

46. The method of any one of claims 1-39, wherein the subject has hirsutism.

47. The method of claim 46, wherein the subject reports their hirsutism has improved following administration of the compound of Formula (I), or a pharmaceutically acceptable salt thereof.

48. The method of any one of claims 1-39, wherein the subject has menstrual dysfunction.

49. The method of claim 48, wherein the subject reports their menstrual dysfunction has improved following administration of the compound of Formula (I), or a pharmaceutically acceptable salt thereof.

50. The method of any one of claims 1-39, wherein the subject has testicular adrenal rests tumor (TART).

51. The method of claim 50, wherein the subject's TART has improved following administration of the compound of Formula (I), or a pharmaceutically acceptable salt thereof.

52. A pharmaceutical composition for use in treating a subject with CAH, wherein the composition comprises a compound of Formula (I), or a pharmaceutically acceptable salt thereof:

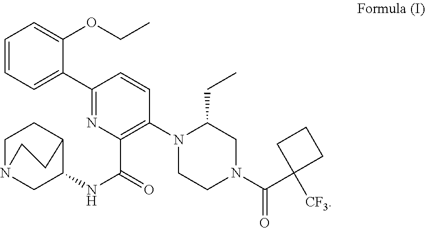 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

53. Use of a compound of Formula (I), or a pharmaceutically acceptable salt thereof, in the manufacture of a medicament for treating a subject with CAH:

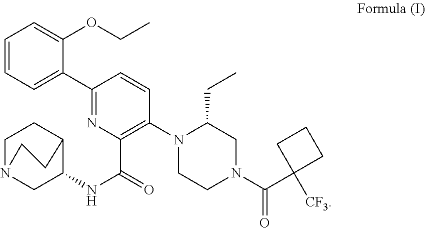 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

54. A pharmaceutical composition for use in treating hyperandrogenism in a subject with CAH, wherein the composition comprises a compound of Formula (I), or a pharmaceutically acceptable salt thereof:

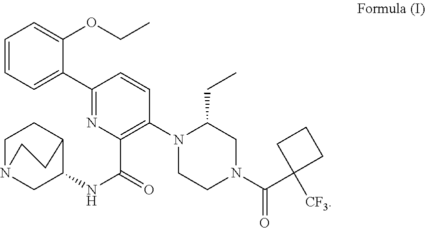 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]

55. Use of a compound of Formula (I), or a pharmaceutically acceptable salt thereof, in the manufacture of a medicament for treating hyperandrogenism in a subject with CAH: 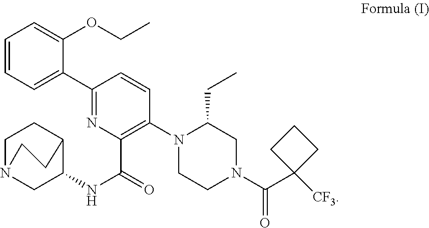 [SMILES: CCOc1ccccc1-c1ccc(N2CCN(C(=O)C3(C(F)(F)F)CCC3)C[C@H]2CC)c(C(=O)N[C@@H]2CN3CCC2CC3)n1]
